(19) United States
(12) Patent Application Publication
(10) Pub. No.: US 2023/0310504 A1
(43) Pub. Date: Oct. 5, 2023

(54) PLATELET MEMBRANE COATED NANOPARTICLES AND USES THEREOF

(71) Applicant: CELLO THERAPEUTICS, INC., San Diego, CA (US)

(72) Inventors: Jie ZHANG, San Diego, CA (US); Brian T. LUK, San Diego, CA (US); Baharak BAHMANI, San Diego, CA (US); Kristofer J. HAUSHALTER, San Diego, CA (US)

(73) Assignee: CELLO THERAPEUTICS, INC., San Diego, CA (US)

(21) Appl. No.: 18/011,668

(22) PCT Filed: Jun. 28, 2021
(86) PCT No.: PCT/US2021/039446

§ 371 Date: Dec. 20, 2022

Related U.S. Application Data
(60) Provisional application No. 63/047,210, filed on Jul. 1, 2020.

(51) Int. Cl. A61K 35/19; A61K 9/51; A61K 31/4745; A61K 31/55; A61K 31/65; A61P 35/00

(52) U.S. Cl. CPC A61K 35/19; A61K 9/5146; A61K 9/5153; A61K 9/5192; A61K 31/4745; A61K 31/55; A61K 31/65; A61P 35/00

(57) ABSTRACT

The present disclosure provides for platelet membrane coated nanoparticles that comprise, inter alia, an immunomodulating agent that is a toll-like receptor (TLR) agonist and/or an upregulator of the opioid growth factor receptor. Compositions, e.g., medicament delivery devices and pharmaceutical compositions, comprising the present nanoparticles are also provided. Uses of the present nanoparticles, including uses of the present nanoparticles for treating or preventing a neoplasm in a subject, are further provided.

### RELATED APPLICATION

The present application claims priority to U.S. Provisional Pat. Application No. 63/047,210, filed on Jul. 1, 2020, the disclosure of which is incorporated herein by reference in its entirety for all purposes.

### TECHNICAL FIELD

The present disclosure provides for platelet membrane coated nanoparticles that comprise, inter alia, an immunomodulating agent that is a toll-like receptor (TLR) agonist and/or an upregulator of the opioid growth factor receptor. Compositions, e.g., medicament delivery devices and pharmaceutical compositions, comprising the present nanoparticles are also provided. Uses of the present nanoparticles, including uses of the present nanoparticles for treating or preventing a neoplasm in a subject, are further provided.

### BACKGROUND

Immunotherapy has emerged as an effective therapeutic approach against cancer that harnesses the power of immune cells in the tumor microenvironment. Some recent approaches, including the use of immune checkpoint inhibitors against cytotoxic T-lymphocyte-associated protein 4 and programmed cell death protein 1^{1,2}, as well as the adoptive transfer of chimeric antigen receptor (CAR) T cells³, have shown considerable promise. Despite the clinical success of such immunotherapies in the treatment of various cancer types⁴⁻⁷, each still has their downsides that need to be overcome. For example, CAR T cell therapy has performed well against certain hematological cancers, but does not fare well against solid tumors⁸. Checkpoint blockade therapy is oftentimes associated with severe systemic side effects and only benefits a subset of patients with tumors that are in the correct immunological state^{9,10}. One promising strategy to further expand the field of immunotherapy is the modulation of the tumor microenvironment via engagement of Toll-like receptors (TLRs) and inhibiting tumor-promoting immune signaling¹¹⁻¹⁴. TLRs are mainly expressed by immune cells, and among them TLR7, an endosomal single-stranded RNA receptor, is predominantly expressed by macrophages, plasmacytoid dendritic cells, natural killer cells, and B cells¹⁵.

Resiquimod (R848), a small molecule immunomodulator, belongs to the TLR⅞ agonist family Upon binding of R848 to TLR⅞, multiple immunomodulatory cytokines, including interleukin 6 (IL-6), IL-12, and interferon α (IFNα) are released, therefore triggering a cascade of signaling pathways that leads to the activation of antigen-presenting cells (APCs) and polarization of T cell responses¹⁶⁻¹⁸. Despite extensive investigations into the role of TLRs in inducing innate immune responses to bacterial and viral pathogens, only recently has attention shifted to their role in anticancer immunosurveillance. TLR⅞ signaling can promote anticancer responses through activation of the central transcription factor nuclear factor κB (NF-ĸB)¹⁹. It has been reported that TLR⅞ therapy leads to the expansion of tumor antigen-specific CD8⁺ T cells, which is important for the development of an effective antitumor immune response^{20,21}.

Although the systemic administration of R848 and other members of the TLR7 agonist family in combination with checkpoint inhibitors has proven advantageous in the treatment of squamous cell carcinoma, colon carcinoma, metastatic melanoma, and pancreatic cancer^{18,22-24}, there are drawbacks limiting their clinical translation. For example, safety concerns were raised when multiple intravenous doses or oral administrations of small molecule TLR7 agonists caused adverse events such as fever, fatigue, headache, and hypertension in patients²⁵⁻²⁸. In addition, some reports suggested that systemic administration of R848 leads to rapid depletion of leukocytes and transient local immune insufficiency²⁹. Consequently, intratumoral injection of TLR7 agonists has been investigated as a more clinically relevant route of administration to address solid tumors³⁰⁻³⁴. The localization of immunostimulatory agents to the tumor microenvironment can convert it from a “cold” to a “hot” state, helping to kickstart antitumor immunity³⁵. In order for intratumoral immunotherapies to be effective, it is necessary to safely confine the immune agonist payloads within the tumor site However, the direct injection of free drug has the potential for systemic exposure due to leakage, whereas targeted nanodelivery platforms are generally designed to be antigen-specific³⁶, limiting their broad applicability

There is a need for improved compositions and methods for treating or preventing various diseases such as a neoplasm in a subject. The present disclosure addresses this and other related needs.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

In one aspect, the present disclosure provides for a nanoparticle, which comprises: a) an inner core comprising a non-cellular material; b) an outer surface comprising a cellular membrane derived from a platelet; and, inter alia, c) an immunomodulating agent that is a toll-like receptor (TLR) agonist and/or an upregulator of the opioid growth factor receptor.

In another aspect, the present disclosure provides for a process for making a nanoparticle comprising: a) contacting an immunomodulating agent that is a toll-like receptor (TLR) agonist and/or an upregulator of the opioid growth factor receptor with a polymer to form an organic phase in an organic solvent; b) contacting said organic phase with an aqueous phase to form a primary emulsion; c) subjecting said primary emulsion to sonication or a high pressure homogenization to form a fine emulsion; d) removing said organic solvent from said fine emulsion to form a nanoparticle comprising said immunomodulating agent and said polymer in said fine emulsion; and e) recovering said nanoparticle from said fine emulsion. Nanoparticles made by the above process are also provided.

Compositions comprising the above nanoparticles and various uses of the above nanoparticles are further provided. In still another aspect, the present disclosure provides for a medicament delivery device, which comprises an effective amount of the above nanoparticle. In yet another aspect, the present disclosure provides for a pharmaceutical composition comprising an effective amount of the above nanoparticle, and a pharmaceutically acceptable carrier or excipient. In yet another aspect, the present disclosure provides for an use of an effective amount of the above nanoparticle for the manufacture of a medicament for treating or preventing a disease or condition in a subject in need. In yet another aspect, the present disclosure provides for a method for treating or preventing a neoplasm in a subject in need comprising administering to said subject an effective amount of the above nanoparticle, medicament delivery device or pharmaceutical composition.

### BRIEF DESCRIPTION OF THE DRAWINGS

FIG. 1 illustrates exemplary nanoparticle characterization. a, Characterization of surface markers on platelet membrane, including phosphatidylserine (PS), P-selectin, GPIbα, and αIIbβ3. b,c, Quantification of pro-thrombotic platelet-activating molecules thrombin (b) and adenosine diphosphate (ADP, c) in platelet-rich plasma (PRP), platelet lysate, and purified platelet membrane (mean + SD). d, Average hydrodynamic diameter and polydispersity index (PDI) of bare nanoparticle (NP) cores, uncoated NP-R848, PNP, and PNP-R848 (mean + SD). e, Zeta potential of bare NP, NP-R848, PNP, and PNP-R848 (mean + SD). f, Transmission electron microscopy visualization of PNP-R848 with uranyl acetate negative staining (scale bar = 50 nm). g, Drug release profile from uncoated NP-R848 and coated PNP-R848 over 3 days.

FIG. 2 illustrates exemplary nanoparticle interaction with tumor cells. a,b, Quantification of binding (a) and uptake (b) of PEG-NP and PNP by various cancer cells (MC38, HT-29, 4T1, and MDA-MB-231) after incubation in vitro (mean + SD; MFI = mean fluorescence intensity). *p < 0.05, **p < 0.01, ***p < 0.001, ****p < 0.0001 (compared with PNP). c, Retention of PEG-NP or PNP over time after intratumoral administration into mice bearing MC38 tumors (mean ± SEM). d, Representative images from the study in (c) at 5 min, 48 h, 96 h, and 168 h (H = high fluorescent signal, L = low fluorescent signal).

FIG. 3 illustrates exemplary in vitro activity and interaction with immune cells. a,b, Dose-dependent response of TLR7 (a) and TLR8 (b) reporter cell lines after incubation with PNP, free R848, and PNP-R848 (mean + SD). c,d, Expression of CD80 (c) and CD86 (d) by bone marrow-derived cells (BMDCs) after incubation with free R848 or PNP-R848. e-g, Dose-dependent secretion of IL-6 (e), TNFα (f), and IL-12p40 (g) by BMDCs after incubation with free R848 and PNP-R848. h,i, Quantification of binding (h) and uptake (i) of PEG-NP and PNP by immune cell subsets (CD45⁺, CD11b⁺, and CD11c⁺) after incubation with BMDCs in vitro (mean + SD; MFI = mean fluorescence intensity). j-l, In vivo uptake of PEG-NP and PNP by the total tumor cell population (j), CD45⁺ cells (k), and CD11c⁺ cells (1) at various timepoints after intratumoral administration (mean + SD). MFI was normalized based on the total cell number.

FIG. 4 illustrates exemplary therapeutic antitumor efficacy in an MC38 murine colorectal adenocarcinoma tumor model. a, Schematic timeline of the efficacy study. b, Average tumor growth kinetics after treatment with free R848, PEG-NP-R848, and PNP-R848 (mean ± SEM). c, Individual tumor growth kinetics after treatment with free R848, PEG-NP-R848, and PNP-R848 (N = naïve challenge, RC = re-challenge). The insets depict the growth kinetics after each re-challenge. d, Progression-free survival (tumor size < 200 mm³) of mice after treatment with free R848, PEG-NP-R848, and PNP-R848. e, Body weight of mice after treatment with free R848, PEG-NP-R848, and PNP-R848 (mean ± SD).

FIG. 5 illustrates exemplary immune response to treatment in MC38 murine colorectal adenocarcinoma tumor-bearing mice. a, Relative expression of MHC-II by CD11b⁺ or CD11c⁺ cells in the DLN from mice treated with free R848 and PNP-R848 (mean + SD). b, Percentage of CD3⁺ cells within the CD45⁺ cell population of the DLN from mice treated with free R848 and PNP-R848 (mean + SD). c, Percentage of CD8⁺ cells within the CD3⁺ cell population of the DLN from mice treated with free R848 and PNP-R848 (mean + SD). d, Proportion of CD4⁺ T cells with the effector memory or central memory phenotypes in the DLN from mice treated with free R848 and PNP-R848 (mean + SD). e, Quantification of CD3⁺, CD4⁺, or CD8⁺ cell density in tumor sections from mice treated with free R848 and PNP-R848 (mean + SD). f, Representative histological sections from the experiment in (e) (scale bar = 100 µm; brown = positive staining). *p < 0.05, **p < 0.01, ***p < 0.001, ****p < 0.0001 (compared with PNP-R848); one-way ANOVA.

FIG. 6 illustrates exemplary therapeutic antitumor efficacy in an MC38 murine colorectal adenocarcinoma tumor model at a reduced R848 dosage. a, Average tumor growth kinetics after treatment with free R848, PEG-NP-R848, and PNP-R848 (mean ± SEM). b, Individual tumor growth kinetics after treatment with free R848, PEG-NP-R848, and PNP-R848 (N = naïve challenge, RC = re-challenge). The insets depict the growth kinetics after each re-challenge. c, Progression-free survival (tumor size < 200 mm³) of mice after treatment with free R848, PEG-NP-R848, and PNP-R848. d, Body weight of mice after treatment with free R848, PEG-NP-R848, and PNP-R848 (mean ± SD).

FIG. 7 illustrates exemplary therapeutic antitumor efficacy of empty nanocarriers in MC38 murine colorectal adenocarcinoma tumor-bearing mice. a, Progression-free survival (tumor size < 200 mm³) of mice after treatment with PEG-NP or PNP without R848 loading (NS = not significant, log-rank test). b, Body weight of mice after treatment with PEG-NP or PNP without R848 loading (mean ± SD).

FIG. 8 illustrates exemplary therapeutic efficacy in combination with chemotherapy in MC38 murine colorectal adenocarcinoma tumor-bearing mice. a, Progression-free survival (tumor size < 200 mm³) of mice after treatment with doxorubicin (DOX) or DOX + PNP-R848. b, Body weight of mice after treatment with doxorubicin (DOX) or DOX + PNP-R848 (mean ± SD).

FIG. 9 illustrates exemplary therapeutic efficacy in a 4T1 murine breast cancer tumor model. a, Schematic timeline of the efficacy study. Tumors were treated with free R848, PEG-NP-R848, or PNP-R848. b, Average tumor growth kinetics after treatment (mean ± SEM). c, Individual tumor growth kinetics after treatment. d, Progression-free survival (tumor size < 200 mm³) of mice after treatment. e, Images of tumors at day 30 post-treatment. f, Average tumor weights at day 30 post-treatment (mean + SD). g, Number of metastatic nodules in the lungs at day 30 post-treatment (mean + SD). *p < 0.05, ***p < 0.001, ****p < 0.0001 (compared with PNP-R848); one-way ANOVA.

### DETAILED DESCRIPTION

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

All publications, including patent documents, scientific articles and databases, referred to in this application are incorporated by reference in their entireties for all purposes to the same extent as if each individual publication were individually incorporated by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

The practice of the provided embodiments will employ, unless otherwise indicated, conventional techniques and descriptions of organic chemistry, polymer technology, molecular biology (including recombinant techniques), cell biology, biochemistry, sequencing technology, immunology, including cancer immunology, and medicine, which are within the skill of those who practice in the art. Such conventional techniques include polypeptide and protein synthesis and modification, polynucleotide synthesis and modification, polymer array synthesis, hybridization and ligation of polynucleotides, detection of hybridization, and nucleotide sequencing. Specific illustrations of suitable techniques can be had by reference to the examples herein. However, other equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, Gabriel, Stephens, Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Ausubel et al. eds., Current Protocols in Molecular Biology (1987); T. Brown ed., Essential Molecular Biology (1991), IRL Press; Goeddel ed., Gene Expression Technology (1991), Academic Press; A. Bothwell et al. eds., Methods for Cloning and Analysis of Eukaryotic Genes (1990), Bartlett Publ.; M. Kriegler, Gene Transfer and Expression (1990), Stockton Press; R. Wu et al. eds., Recombinant DNA Methodology (1989), Academic Press; M. McPherson et al., PCR: A Practical Approach (1991), IRL Press at Oxford University Press; Stryer, Biochemistry (4th Ed.) (1995), W. H. Freeman, New York N.Y.; Gait, Oligonucleotide Synthesis: A Practical Approach (2002), IRL Press, London; Nelson and Cox, Lehninger, Principles of Biochemistry (2000) 3rd Ed., W. H. Freeman Pub., New York, N.Y.; Berg, et al., Biochemistry (2002) 5th Ed., W. H. Freeman Pub., New York, N.Y., all of which are herein incorporated in their entireties by reference for all purposes.

To facilitate understanding of the invention, a number of terms and abbreviations as used herein are defined below as follows.

### A. Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

Cellular Membrane: The term “cellular membrane” as used herein refers to a biological membrane enclosing or separating structure acting as a selective barrier, within or around a cell. The cellular membrane is selectively permeable to ions and organic molecules and controls the movement of substances in and out of cells. The cellular membrane comprises a phospholipid uni- or bilayer, and optionally associated proteins and carbohydrates. As used herein, the cellular membrane refers to a membrane obtained from a naturally occurring biological membrane of a cell or cellular organelles, or one derived therefrom. As used herein, the term “naturally occurring” refers to one existing in nature. As used herein, the term “derived therefrom” refers to any subsequent modification of the natural membrane, such as isolating the cellular membrane, creating portions or fragments of the membrane, removing and/or adding certain components, such as lipid, protein or carbohydrates, from or into the membrane taken from a cell or a cellular organelle. A membrane can be derived from a naturally occurring membrane by any suitable methods. For example, a membrane can be prepared or isolated from a cell and the prepared or isolated membrane can be combined with other substances or materials to form a derived membrane. In another example, a cell can be recombinantly engineered to produce “non-natural” substances that are incorporated into its membrane in vivo, and the cellular membrane can be prepared or isolated from the cell to form a derived membrane.

In various embodiments, the cellular membrane covering either of the unilamellar or multilamellar nanoparticles can be further modified to be saturated or unsaturated with other lipid components, such as cholesterol, free fatty acids, and phospholipids, also can include endogenous or added proteins and carbohydrates, such as cellular surface antigen. In such cases, an excess amount of the other lipid components can be added to the membrane wall which will shed until the concentration in the membrane wall reaches equilibrium, which can be dependent upon the nanoparticle environment. Membranes may also comprise other agents that may or may not increase an activity of the nanoparticle. In other examples, functional groups such as antibodies and aptamers can be added to the outer surface of the membrane to enhance site targeting, such as to cell surface epitopes found in cancer cells. The membrane of the nanoparticles can also comprise particles that can be biodegradable, cationic nanoparticles including, but not limited to, gold, silver, and synthetic nanoparticles.

Synthetic or artificial membrane: As used herein, the term “synthetic membrane” or “artificial membrane” refers to a man-made membrane that is produced from organic material, such as polymers and liquids, as well as inorganic materials. A wide variety of synthetic membranes are well known in the art.

Nanoparticle: In some embodiments, the term “nanoparticle” as used herein refers to nanostructure, particles, vesicles, or fragments thereof having at least one dimension (e.g., height, length, width, or diameter) of between about 1 nm and about 10 µm. For systemic use, an average diameter of about 30 nm to about 500 nm, or about 30 nm to about 300 nm, or about 50 nm to about 250 nm may be preferred. The term “nanostructure” includes, but is not necessarily limited to, particles and engineered features. The particles and engineered features can have, for example, a regular or irregular shape. Such particles are also referred to as nanoparticles. The nanoparticles can be composed of organic materials or other materials, and can alternatively be implemented with porous particles. The layer of nanoparticles can be implemented with nanoparticles in a monolayer or with a layer having agglomerations of nanoparticles. In some embodiments, the nanoparticle comprising or consisting of an interior compartment (or an inner core) can be covered by an outer surface (or shell) comprising the membrane as discussed herein. The disclosure contemplates any nanoparticles now known and later developed that can be coated with the membrane described herein.

Pharmaceutically active: The term “pharmaceutically active” as used herein refers to the beneficial biological activity of a substance on living matter and, in particular, on cells and tissues of the human body. A “pharmaceutically active agent” or “drug” is a substance that is pharmaceutically active and a “pharmaceutically active ingredient” (API) is the pharmaceutically active substance in a drug.

Pharmaceutically acceptable: The term “pharmaceutically acceptable” as used herein means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

Pharmaceutically acceptable salt: The term “pharmaceutically acceptable salt” as used herein refers to acid addition salts or base addition salts of the compounds, such as the multi-drug conjugates, in the present disclosure. A pharmaceutically acceptable salt is any salt which retains the activity of the parent nanoparticle or compound and does not impart any deleterious or undesirable effect on a subject to whom it is administered and in the context in which it is administered. Pharmaceutically acceptable salts may be derived from amino acids including, but not limited to, cysteine. Methods for producing compounds as salts are known to those of skill in the art (see, for example, Stahl et al., Handbook of Pharmaceutical Salts: Properties, Selection, and Use, Wiley-VCH; Verlag Helvetica Chimica Acta, Zurich, 2002; Berge et al., J Pharm. Sci. 66: 1, 1977). In some embodiments, a “pharmaceutically acceptable salt” is intended to mean a salt of a free acid or base of a nanoparticle or compound represented herein that is non-toxic, biologically tolerable, or otherwise biologically suitable for administration to the subject. See, generally, Berge, et al., J. Pharm. Sci., 1977, 66, 1-19. Preferred pharmaceutically acceptable salts are those that are pharmacologically effective and suitable for contact with the tissues of subjects without undue toxicity, irritation, or allergic response. A nanoparticle or compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosul fates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne- 1,4-dioates, hexyne- 1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene- 1 -sulfonates, naphthalene-2-sulfonates, phenylacetates, phenylpropionates, phenylbutyrates, citrates, lactates, [gamma]-hydroxybutyrates, glycolates, tartrates, and mandelates.

Pharmaceutically acceptable carrier: The term “pharmaceutically acceptable carrier” as used herein refers to an excipient, diluent, preservative, solubilizer, emulsifier, adjuvant, and/or vehicle with which a nanoparticle or compound, such as a multi-drug conjugate, is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy. 20‴ ed., (Lippincott, Williams & Wilkins 2003). Except insofar as any conventional media or agent is incompatible with the active compound, such use in the compositions is contemplated.

Phospholipid: The term “phospholipid”, as used herein, refers to any of numerous lipids contain a diglyceride, a phosphate group, and a simple organic molecule such as choline. Examples of phospholipids include, but are not limited to, Phosphatide acid (phosphatidate) (PA), Phosphatidylethanolamine (cephalin) (PE), Phosphatidylcholine (lecithin) (PC), Phosphatidylserine (PS), and Phosphoinositides which include, but are not limited to, Phosphatidylinositol (PI), Phosphatidylinositol phosphate (PIP), Phosphatidylinositol bisphosphate (PIP2) and Phosphatidylinositol triphosphate (P1P3). Additional examples of PC include DDPC, DLPC, DMPC, DPPC, DSPC, DOPC, POPC, DRPC, and DEPC as defined in the art.

Therapeutically Effective Amount: As used herein, the term “therapeutically effective amount” refers to those amounts that, when administered to a particular subject in view of the nature and severity of that subject’s disease or condition, will have a desired therapeutic effect, e.g., an amount which will cure, prevent, inhibit, or at least partially arrest or partially prevent a target disease or condition. More specific embodiments are included in the Pharmaceutical Preparations and Methods of Administration section below. In some embodiments, the term “therapeutically effective amount” or “effective amount” refers to an amount of a therapeutic agent that when administered alone or in combination with an additional therapeutic agent to a cell, tissue, or subject is effective to prevent or ameliorate the disease or condition such as a tumor or cancer, or the progression of the disease or condition. A therapeutically effective dose further refers to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously.

“Treating” or “treatment” or “alleviation” refers to therapeutic treatment wherein the object is to slow down (lessen) if not cure the targeted pathologic condition or disorder or prevent recurrence of the condition. A subject is successfully “treated” if, after receiving a therapeutic amount of a therapeutic agent, the subject shows observable and/or measurable reduction in or absence of one or more signs and symptoms of the particular disease. Reduction of the signs or symptoms of a disease may also be felt by the patient. A patient is also considered treated if the patient experiences stable disease. In some embodiments, treatment with a therapeutic agent is effective to result in the patients being disease-free 3 months after treatment, preferably 6 months, more preferably one year, even more preferably 2 or more years post treatment. These parameters for assessing successful treatment and improvement in the disease are readily measurable by routine procedures familiar to a physician of appropriate skill in the art.

As used herein, “preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition.

The term “combination” refers to either a fixed combination in one dosage unit form, or a kit of parts for the combined administration where a nanoparticle or compound and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a nanoparticle or compound and a combination partner, are both administered to a patient simultaneously in the form of a single entity or dosage. The term “non-fixed combination” means that the active ingredients, e.g., a nanoparticle or compound and a combination partner, are both administered to a patient as separate entities either simultaneously, concurrently or sequentially with no specific time limits, wherein such administration provides therapeutically effective levels of the two moieties or compounds in the body of the patient. The latter also applies to cocktail therapy, e.g., the administration of three or more active ingredients.

As used herein, a subject in need refers to an animal, a non-human mammal or a human. As used herein, “animals” include a pet, a farm animal, an economic animal, a sport animal and an experimental animal, such as a cat, a dog, a horse, a cow, an ox, a pig, a donkey, a sheep, a lamb, a goat, a mouse, a rabbit, a chicken, a duck, a goose, a primate, including a monkey and a chimpanzee. A “subject” as used herein refers to an organism, or a part or component of the organism, to which the provided compositions, methods, kits, devices, and systems can be administered or applied. For example, the subject can be a mammal or a cell, a tissue, an organ, or a part of the mammal. As used herein, “mammal” refers to any of the mammalian class of species, preferably human (including humans, human subjects, or human patients). Subjects and mammals include, but are not limited to, farm animals, sport animals, pets, primates, horses, dogs, cats, and rodents such as mice and rats.

As used herein the term “sample” refers to anything which may contain a target molecule for which analysis is desired, including a biological sample. As used herein, a “biological sample” can refer to any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine, sweat, semen, stool, sputum, tears, mucus, amniotic fluid or the like, an effusion, a bone marrow sample, ascitic fluid, pelvic wash fluid, pleural fluid, spinal fluid, lymph, ocular fluid, extract of nasal, throat or genital swab, cell suspension from digested tissue, or extract of fecal material, and tissue and organ samples from humans, animals, e.g., non-human mammals, and plants and processed samples derived therefrom.

As used herein, “disease or disorder” refers to a pathological condition in an organism resulting from, e.g., infection or genetic defect or other causes, and characterized by identifiable symptoms.

It is understood that aspects and embodiments of the invention described herein include “consisting” and/or “consisting essentially of′ aspects and embodiments.

The term “average” as used herein refers to either a mean or a median, or any value used to approximate the mean or the median, unless the context clearly indicates otherwise.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present invention will become apparent from the following specification taken in conjunction with the accompanying drawings.

### B. Platelet Membrane Coated Nanoparticles and Compositions Comprising the Same

In one aspect, the present disclosure provides for a nanoparticle, which comprises: a) an inner core comprising a non-cellular material; b) an outer surface comprising a cellular membrane derived from a platelet; and c) an immunomodulating agent that is a toll-like receptor (TLR) agonist and/or an upregulator of the opioid growth factor receptor.

The inner core of the present nanoparticles can comprise any suitable substance or material. For example, the inner core of the present nanoparticles can comprise a polymer. The inner core of the present nanoparticles can comprise any suitable polymer. In some embodiments, the polymer is a biocompatible and/or biodegradable polymer. In some embodiments, the polymer is a homopolymer. In some embodiments, the homopolymer can comprise lactic acid units, e.g., lactic acid units comprising poly-L-lactic acid, poly-D-lactic acid, poly-D,L-lactic acid, poly-L-lactide, poly-D-lactide or poly-D,L-lactide units. In some embodiments, the polymer is a copolymer. The copolymer can comprise lactic acid and glycolic acid units, e.g., lactic acid and glycolic acid units comprising poly(lactic-co-glycolic) acid and poly(lactide-co-glycolide). In some embodiments, the inner core of the present nanoparticles can comprise a biocompatible or a synthetic material selected from the group consisting of poly(lactic-co-glycolic acid) (PLGA), polylactic acid (PLA), polyglycolic acid (PGA), polycaprolactone (PCL), polylysine, and polyglutamic acid.

The outer surface of the present nanoparticles can comprise any suitable membrane derived from a platelet. For example, the outer surface of the present nanoparticles can comprise a plasma membrane derived from a platelet. In another example, the outer surface of the present nanoparticles can comprise an intracellular membrane derived from a platelet. In still another example, the outer surface of the present nanoparticles can comprise a naturally occurring cellular membrane derived from a platelet. In yet another example, the outer surface of the present nanoparticles can comprise a modified membrane derived from a platelet. In yet another example, the outer surface of the present nanoparticles can comprise a hybrid membrane comprising a naturally occurring cellular membrane derived from a platelet and a synthetic membrane.

The present nanoparticles can comprise any suitable immunomodulating agent that is a toll-like receptor (TLR) agonist and/or an upregulator of the opioid growth factor receptor. For example, the present nanoparticles can comprise an immunomodulating agent that is a small molecule, a polynucleotide, a nucleic acid, a polypeptide, a protein, a peptide, a lipid, a carbohydrate, a hormone, a metal, and/or a combination or a complex thereof. In some embodiments, the present nanoparticles can comprise an immunomodulating agent that is a toll-like receptor (TLR) agonist. The TLR agonist can target any suitable TLR. For example, the TLR agonist can target TLR ½, TLR 2, TLR 3, TLR 4, TLR 5, TLR ⅚, TLR 7, TLR 8, TLR 9, or TLR 10. In some embodiments, the present nanoparticles can comprise an immunomodulating agent that is an upregulator of the opioid growth factor receptor.

In some embodiments, the present nanoparticles can comprise an immunomodulating agent that is resiquimod, imiquimod, or motolimod. In specific embodiments, the present nanoparticles can comprise an immunomodulating agent that is resiquimod.

In the present nanoparticles, the immunomodulating agent can be located at any suitable location. For example, the immunomodulating agent can be located within or on the inner core, between the inner core and the outer surface, or within or on the outer surface.

The release of the immunomodulating agent from the present nanoparticles can be triggered by any suitable ways or mechanisms. For example, the release of the immunomodulating agent can be triggered by a contact between the nanoparticle and a target cell, tissue, organ or subject, or by a change of a physical parameter surrounding the nanoparticle.

The interior of the present nanoparticles can have any suitable hydrophobicity. For example, the interior of the present nanoparticles can be more hydrophobic than the outer surface of the nanoparticle. In some embodiments, the immunomodulating agent is hydrophobic and located within the hydrophobic or more hydrophobic interior of the present nanoparticles.

In some embodiments, the inner core of the present nanoparticles supports the outer surface of the present nanoparticles.

The present nanoparticles can have any suitable size or diameter. For example, the present nanoparticles can have a diameter from about 10 nm to about 10 µm, e.g., a diameter at about 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 250 nm, 300 nm, 350 nm, 400 nm, 450 nm, 500 nm, 600 nm, 700 nm, 800 nm, 900 nm, 1 µm, 2 µm, 3 µm, 4 µm, 5 µm, 6 µm, 7 µm, 8 µm, 9 µm, 10 µm, or any subrange thereof. In some embodiments, the present nanoparticles have a diameter from about 50 nm to about 1 µm, or any subrange thereof. In some embodiments, the present nanoparticles have a diameter from about 70 nm to about 150 nm, or any subrange thereof.

The present nanoparticles can have any suitable shape, including but not limited to, sphere, square, rectangle, triangle, circular disc, cube-like shape, cube, rectangular parallelepiped (cuboid), cone, cylinder, prism, pyramid, right-angled circular cylinder and other regular or irregular shape. In some embodiments, the present nanoparticles have a substantially spherical configuration or a non-spherical configuration.

In some embodiments, the present nanoparticles substantially lack constituents of the platelet from which the cellular membrane, e.g., a plasma membrane, is derived. For example, the present nanoparticles can lack about 10%, 20%, 30%, 40%, 50%, 55%, 60%, 65%, 70%, 75%, 80%, 85%, 90%, 91%, 92%, 93%, 94%, 95%, 96%, 97%, 98%, 99%, or 100% of the constituents of the platelet from which the cellular membrane, e.g., a plasma membrane, is derived.

In some embodiments, the present nanoparticles substantially maintain natural structural integrity or activity of the cellular membrane, e.g., a plasma membrane, or the constituents of the cellular membrane. For example, the present nanoparticles can retain about 10%, 20%, 30%, 40%, 50%, 55%, 60%, 65%, 70%, 75%, 80%, 85%, 90%, 91%, 92%, 93%, 94%, 95%, 96%, 97%, 98%, 99%, or 100% of the natural structural integrity. In some embodiments, the present nanoparticles substantially maintain natural structural integrity of the cellular membrane or the constituents of the cellular membrane including primary, secondary, tertiary and/or quaternary structure of the cellular membrane, or the constituents of the cellular membrane. In some embodiments, the present nanoparticles substantially maintain activity of the cellular membrane or the constituents of the cellular membrane including binding activity, receptor activity and/or enzymatic activity of the cellular membrane, or the constituents of the cellular membrane.

In some embodiments, the present nanoparticles are biocompatible or biodegradable. For example, the inner core of the present nanoparticles can comprise a biocompatible or biodegradable material and the outer surface of the present nanoparticles comprises a plasma membrane derived from a platelet. In another example, the interior compartment (or an inner core) only comprises biocompatible or biodegradable material, or does not comprise any material that is not biocompatible or biodegradable.

In some embodiments, the present nanoparticle comprises an inner core comprising a copolymer comprising lactic acid and glycolic acid units, e.g., poly(lactic-co-glycolic) acid and poly(lactide-co-glycolide), an outer surface comprising a plasma membrane derived from a platelet, and resiquimod, e.g., resiquimod (R-848, S-27609).

The present nanoparticles can have any suitable half-life or half-life in a solid tumor. In some embodiments, the present nanoparticles have a half-life in a solid tumor for about 48 hours to about 72 hours, e.g., a half-life in a solid tumor for about 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, 72 hours, or any subrange thereof.

In some embodiments, the present nanoparticles substantially lack immunogenicity to a subject, a mammal, a non-human mammal or a human, to which the present nanoparticles are configured to administer. For example, the cellular membrane can be derived from a platelet from the same species of the subject. In another example, the subject is a human and the cellular membrane is derived from a human platelet. In some embodiments, the cellular membrane can be derived from a platelet of the subject to be treated. For example, the cellular membrane can be derived from a platelet of the human to be treated.

The outer surface of the present nanoparticles can comprise a hybrid membrane comprising a cellular membrane derived from a platelet and a synthetic membrane. In some embodiments, the outer surface of the nanoparticles can comprise a hybrid membrane comprising at least about 5% (w/w), 6% (w/w), 7% (w/w), 8% (w/w), 9% (w/w), 10% (w/w), 20% (w/w), 30% (w/w), 40% (w/w), 50% (w/w), 60% (w/w), 70% (w/w), 80% (w/w), 90% (w/w), 91 % (w/w), 92% (w/w), 93% (w/w), 94% (w/w), 95% (w/w), 96% (w/w), 97% (w/w), 98% (w/w), 99% (w/w) of a cellular membrane derived from a platelet. In other embodiments, the outer surface of the present nanoparticles can comprise a hybrid membrane comprising at least about 1% (w/w), 2% (w/w), 3% (w/w), 4% (w/w), 5% (w/w), 6% (w/w), 7% (w/w), 8% (w/w), 9% (w/w), 10% (w/w), 20% (w/w), 30% (w/w), 40% (w/w), 50% (w/w), 60% (w/w), 70% (w/w), 80% (w/w), 90% (w/w), 91% (w/w), 92% (w/w), 93% (w/w), 94% (w/w), 95% (w/w) of a synthetic membrane. For example, the outer surface of the present nanoparticles can comprise a hybrid membrane comprising about 5-10% (w/w) of a cellular membrane and about 95-99% (w/w) of a synthetic membrane, about 11-25% (w/w) of a cellular membrane and about 75-89% (w/w) of a synthetic membrane, about 50% (w/w) of a cellular membrane and about 50% (w/w) of a synthetic membrane, about 51-75% (w/w) of a cellular membrane and about 49-25% (w/w) of a synthetic membrane, or about 90-99% (w/w) of a cellular membrane and about 1-10% (w/w) of a synthetic membrane.

The present nanoparticles can comprise any suitable amount or level of a therapeutic immunomodulatory agent that is a toll-like receptor (TLR) agonist and/or an upregulator of the opioid growth factor receptor. For example, the present nanoparticles can comprise from about 1 weight percent to about 10 weight percent of the therapeutic immunomodulatory agent, e.g., about 1% (w/w), 2% (w/w), 3% (w/w), 4% (w/w), 5% (w/w), 6% (w/w), 7% (w/w), 8% (w/w), 9% (w/w), 10% (w/w), or any subrange thereof, of the therapeutic immunomodulatory agent.

The present nanoparticles can comprise any suitable amount or level of a biocompatible polymer. For example, the present nanoparticles can comprise from about 50 weight percent to about 99 weight percent of a biocompatible polymer, e.g., about 50% (w/w), 55% (w/w), 60% (w/w), 65% (w/w), 70% (w/w), 75% (w/w), 80% (w/w), 85% (w/w), 90% (w/w), 95% (w/w), 95% (w/w), 96% (w/w), 97% (w/w), 98% (w/w), 99% (w/w), or any subrange thereof, of a biocompatible polymer.

The present nanoparticles can comprise any suitable amount or level of a cellular membrane derived from a platelet. For example, the present nanoparticles can comprise from about 20 weight percent to about 50 weight percent of a cellular membrane derived from a platelet, e.g., about 20% (w/w), 25% (w/w), 30% (w/w), 35% (w/w), 40% (w/w), 45% (w/w), 50% (w/w), or any subrange thereof, of a cellular membrane derived from a platelet.

In some embodiments, the present nanoparticles can comprise about 1 to about 10 weight percent of a therapeutic immunomodulatory agent, about 50 to about 99 weight percent of a biocompatible polymer, and about 20 to about 50 weight percent of a cellular membrane derived from a platelet. In some embodiments, the present nanoparticles can comprise about 1 to about 10 weight percent of resiquimod, about 50 to about 99 weight percent of a biocompatible polymer, and about 20 to about 50 weight percent of a plasma membrane derived from a platelet.

In another aspect, the present disclosure provides for a process for making a nanoparticle comprising steps: a) contacting an immunomodulating agent that is a toll-like receptor (TLR) agonist and/or an upregulator of the opioid growth factor receptor with a polymer to form an organic phase in an organic solvent; b) contacting said organic phase with an aqueous phase to form a primary emulsion; c) subjecting said primary emulsion to sonication or a high pressure homogenization to form a fine emulsion; d) removing said organic solvent from said fine emulsion to form a nanoparticle comprising said immunomodulating agent and said polymer in said fine emulsion; and e) recovering said nanoparticle from said fine emulsion.

Any suitable toll-like receptor (TLR) agonist and/or an upregulator of the opioid growth factor receptor can be used in the present processes. For example, the immunomodulating agent can be resiquimod, imiquimod, or motolimod. In some embodiments, the immunomodulating agent is resiquimod.

Any suitable polymer can be used in the present processes. For example, a homopolymer or copolymer can be used in the present processes. In some embodiments, a homopolymer or copolymer that comprises lactic acid and/or glycolic acid units can be used in the present processes.

Any suitable organic phase can be used in the present processes. For example, an organic phase comprising acetonitrile, tetrahydrofuran, ethyl acetate, isopropyl alcohol, isopropyl acetate, dimethylformamide, methylene chloride, chloroform, acetone, benzyl alcohol, sodium cholate, Tween 80, or the like, or a combination thereof, can be used in the present processes. In some embodiments, an organic phase comprising benzyl alcohol, ethyl acetate, dichloromethane, or a combination thereof, can be used in the present processes.

An organic phase comprising any suitable amount or level of the polymer and the immunomodulating agent can be used in the present processes. For example, an organic phase comprising about 5 to about 10% weight solids of the polymer and the immunomodulating agent can be used in the present processes. In some embodiments, an organic phase comprising about 5% (w/w), 6% (w/w), 7% (w/w), 8% (w/w), 9% (w/w), 10% (w/w), or any subrange thereof, of the polymer and the immunomodulating agent can be used in the present processes.

Any suitable aqueous solution comprising water can be used in the present processes. In some embodiments, an aqueous solution comprising water in combination with one or more of sodium cholate, tris(hydroxymethyl)aminomethane hydrochloride, ethyl acetate, and benzyl alcohol can be used in the present processes.

The primary emulsion can comprise any suitable amount or level of the polymer and the immunomodulating agent. For example, the primary emulsion can comprise about 1 to about 10% weight solids of the polymer and the immunomodulating agent. In some embodiments, the primary emulsion can comprise about 1% (w/w), 2% (w/w), 3% (w/w), 4% (w/w), 5% (w/w), 6% (w/w), 7% (w/w), 8% (w/w), 9% (w/w), 10% (w/w), or any subrange thereof, of the polymer and the immunomodulating agent.

In the present processes, step b) can be conducted using any suitable procedure or in any suitable manner. For example, in the present processes, step b) can be conducted by contacting said organic phase with an aqueous phase using simple mixing, high pressure homogenization, probe sonication, stirring, or homogenization via rotor stator to form a primary emulsion.

In the present processes, step c) can be conducted using any suitable procedure or in any suitable manner. For example, in the present processes, step c) can comprise subjecting the primary emulsion to one or more passes through a homogenizer. In another example, in the present processes, step c) can comprise subjecting the primary emulsion to a high pressure homogenization using a pressure from about 5,000 psi to about 15,000 psi. In some embodiments, in the present processes, step c) can comprise subjecting the primary emulsion to a high pressure homogenization using a pressure from about 5,000 psi, 6,000 psi, 7,000 psi, 8,000 psi, 9,000 psi, 10,000 psi, 11,000 psi, 12,000 psi, 13,000 psi, 14,000 psi, 15,000 psi, or any subrange thereof.

In the present processes, step d) can be conducted using any suitable procedure or in any suitable manner. For example, in the present processes, step d) can comprise quenching the fine emulsion by diluting the fine emulsion into a cold aqueous solution or water to a concentration sufficient to dissolve all organic solvent in the fine emulsion to form a quenched phase. The quenching can be conducted at any suitable temperature. For example, the quenching can be conducted at a temperature from about 1° C. to about 5° C. In some embodiments, the quenching for example, can be conducted at a temperature from about 1° C., 2° C., 3° C., 4° C., 5° C., or any subrange thereof. In another example, in the present processes, the step d) can comprise recovering the nanoparticle from the fine emulsion via centrifugation, filtration, ultrafiltration or diafiltration. The filtration, ultrafiltration or diafiltration can be conducted using any suitable membrane. In some embodiments, the filtration, ultrafiltration or diafiltration can be conducted using a membrane with molecular weight cut-offs from about 100 kDa to about 500 kDa., e.g., using a membrane with molecular weight cut-offs at about 100 kDa, 200 kDa, 300 kDa, 400 kDa, 500 kDa., or any subrange thereof.

A nanoparticle made by the above process is provided.

In some embodiments, the present process can further comprise contacting the nanoparticle with a cellular membrane derived from a platelet to form a platelet membrane coated nanoparticle. Any suitable technique or procedure can be used for contacting the nanoparticle with a cellular membrane derived from a platelet to form a platelet membrane coated nanoparticle. For example, the techniques or procedures disclosed and/or claimed in WO 2013/052167 A2, US 2013/0337066 A1, WO 2017/087897 A1, US 2019/0382539 A1, WO 2020/112694 A1, and WO 2020/112694 A9 can be used. A nanoparticle made by the above process is also provided.

In still another aspect, the present disclosure provides for a medicament delivery device, which comprises an effective amount of the above nanoparticle(s). In some embodiments, the present medicament delivery device can further comprise another (or a second) active ingredient, or a medically or pharmaceutically acceptable carrier or excipient. The present medicament delivery device can further comprise any suitable other active ingredient. In some embodiments, the present medicament delivery device can further comprise the other active ingredient that is an anti-neoplastic agent or substance, e.g., doxycycline or doxorubicin. In some embodiments, the present medicament delivery device does not further comprise another active ingredient, e.g., does not further comprise another anti-neoplastic agent or substance, e.g., doxycycline or doxorubicin.

In yet another aspect, the present disclosure provides for a pharmaceutical composition comprising an effective amount of the above nanoparticle, and a pharmaceutically acceptable carrier or excipient. In some embodiments, the present pharmaceutical composition can further comprise another (or a second) active ingredient. The present pharmaceutical composition can further comprise any suitable another active ingredient. In some embodiments, the present pharmaceutical composition can further comprise any suitable another active ingredient that is an anti-neoplastic agent or substance, e.g., doxycycline or doxorubicin. In some embodiments, the present pharmaceutical composition does not further comprise another active ingredient, e.g., does not further comprise another anti-neoplastic agent or substance, e.g., doxycycline or doxorubicin.

In yet another aspect, the present disclosure provides for an use of an effective amount of the above nanoparticle for the manufacture of a medicament for treating or preventing a disease or condition in a subject in need.

The present nanoparticle(s) can be configured for any suitable uses for applications. In some embodiments, the present nanoparticle(s) can be configured for treating or preventing a neoplasm in a subject in need. In some embodiments, the present nanoparticle(s) can be configured for treating or preventing a solid tumor or cancer in a subject in need.

The present nanoparticle(s) can be used alone. In some embodiments, the present nanoparticle(s) can be used in combination with another active substance, e.g., another anti-neoplastic agent or substance. In some embodiments, the present nanoparticle(s) can be used alone without another active substance, e.g., another anti-neoplastic agent or substance.

### C. Methods for Treating or Preventing a Neoplasm in a Subject

In yet another aspect, the present disclosure provides for a method for treating or preventing a neoplasm in a subject in need comprising administering to said subject an effective amount of the above nanoparticle, medicament delivery device, or pharmaceutical composition.

The present methods can be used for any suitable purpose or applications. For example, the present methods can be used for preventing a neoplasm in a subject. In another example, the present methods can be used for treating a neoplasm in a subject.

The nanoparticle used in the present methods can comprise any suitable cellular membrane derived from a platelet. For example, the nanoparticle used in the present methods can comprise a cellular membrane derived from a platelet of the same species of the subject or derived from a platelet of the subject to be treated.

The present methods can be used for treating or preventing a neoplasm in any suitable subject. For example, the present methods can be used for treating or preventing a neoplasm in a non-human subject or mammal. In another example, the present methods can be used for treating or preventing a neoplasm in a human.

Any suitable toll-like receptor (TLR) agonist and/or an upregulator of the opioid growth factor receptor can be used in the present methods. For example, the immunomodulating agent can be resiquimod, imiquimod, or motolimod. In some embodiments, the immunomodulating agent is resiquimod.

The present methods can be used for treating or preventing any suitable neoplasm in a subject. For example, the present methods can be used for treating or preventing a lymphoma, a leukemia, a brain cancer, glioma/glioblastoma (GBM), a multiple myeloma, a pancreatic cancer, a liver cancer, a stomach cancer, a breast cancer, a kidney cancer, a lung cancer, non-small cell lung cancer (NSCLC), a colorectal cancer, a colon cancer, a prostate cancer, an ovarian cancer, a cervical cancer, a skin cancer, an esophageal cancer, or a head and neck cancer in a subject. In some embodiments, the present methods can be used for treating or preventing a solid cancer or tumor in a subject.

In the present methods, the nanoparticle, medicament delivery or pharmaceutical composition can be administered to a subject via any suitable route. For example, in the present methods, the nanoparticle, medicament delivery or pharmaceutical composition can be administered to a subject via an intratumoral, oral, nasal, inhalational, parenteral, intravenous, intraperitoneal, subcutaneous, intramuscular, intradermal, topical, or rectal route. In some embodiments, in the present methods, the nanoparticle, medicament delivery or pharmaceutical composition can be administered intratumorally or in situ to a cancer or tumor site in a subject. In some embodiments, in the present methods, the nanoparticle, medicament delivery or pharmaceutical composition can be administered intratumorally or in situ to a solid cancer or tumor site in a subject.

In the present methods, the nanoparticle, medicament delivery or pharmaceutical composition can be administered to a subject at any suitable dosage or dosage regimen. For example, in the present methods, the nanoparticle, medicament delivery or pharmaceutical composition can be administered to a subject at a dosage from about 0.01 mg/kg to about 0.5 mg/kg. In some embodiments, in the present methods, the nanoparticle, medicament delivery or pharmaceutical composition can be administered to a subject at a dosage at about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, or any subrange thereof.

In some embodiments, in the present methods, the nanoparticle, medicament delivery or pharmaceutical composition can be administered to a subject in combination with another anti-neoplastic agent or substance, e.g., doxycycline or doxorubicin. In some embodiments, in the present methods, the nanoparticle, medicament delivery or pharmaceutical composition can be administered to a subject alone without another anti-neoplastic agent or substance, e.g., doxycycline or doxorubicin.

In some embodiments, in the present methods, the nanoparticle, medicament delivery or pharmaceutical composition can be administered to a subject that has been treated with anti-neoplastic agent or substance, e.g., doxycycline or doxorubicin. In some embodiments, in the present methods, the nanoparticle, medicament delivery or pharmaceutical composition can be administered to a subject that has not been treated with another anti-neoplastic agent or substance, e.g., doxycycline or doxorubicin.

The present methods can be used in any suitable manner. In some embodiments, in the present methods, the nanoparticle, medicament delivery or pharmaceutical composition can be administered to a subject as a first line of treatment. In some embodiments, in the present methods, the nanoparticle, medicament delivery or pharmaceutical composition can be administered to a subject with relapse neoplasm, e.g., a relapse solid cancer or tumor.

In the present methods, the administered nanoparticle, medicament delivery or pharmaceutical composition can be retained within a solid cancer or tumor in a subject for any suitable time period. For example, in the present methods, at least about 50% of the administered nanoparticle, medicament delivery or pharmaceutical composition can be retained within a solid cancer or tumor for at least about 40 hours. In some embodiments, in the present methods, at least about 50%, 60%, 70%, 80%, 90%, 95% or higher level of the administered nanoparticle, medicament delivery or pharmaceutical composition can be retained within a solid cancer or tumor for at least about 40 hours in a subject, e.g., a mouse or a human.

The present methods can be used to achieve any suitable survival rate. For example, the present methods can be used to achieve an overall survival rate of at least about 80% in the treated subjects for at least about three months. In some embodiments, the present methods can be used to achieve a survival rate of at least about 80%, 85%, 90%, 95%, or higher level in a subject, e.g., a mouse or a human.

### D. Pharmaceutical Compositions and Administration Routes

The pharmaceutical compositions comprising the nanoparticles, alone or in combination with other active ingredient(s), described herein may further comprise one or more pharmaceutically-acceptable excipients. A pharmaceutically-acceptable excipient is a substance that is non-toxic and otherwise biologically suitable for administration to a subject. Such excipients facilitate administration of the nanoparticles, alone or in combination with other active ingredient(s), described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the various embodiments are sterile compositions. Pharmaceutical compositions may be prepared using compounding techniques known or that become available to those skilled in the art.

Sterile compositions are within the present disclosure, including compositions that are in accord with national and local regulations governing such compositions.

The pharmaceutical compositions and the nanoparticles, alone or in combination with other active ingredient(s), described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. The nanoparticles, alone or in combination with other active ingredient(s), described herein, and preferably in the form of a pharmaceutical composition, may be administered by a suitable route of delivery, such as oral, intratumoral, rectal, nasal, topical, or ocular routes, or by inhalation or other parenteral route(s). In some embodiments, the compositions are formulated for intratumoral, intravenous or oral administration.

For oral administration, the nanoparticles, alone or in combination with another active ingredient, may be provided in a solid form, such as a tablet or capsule, or as a solution, emulsion, or suspension. To prepare the oral compositions, the nanoparticles, alone or in combination with other active ingredient(s), may be formulated to yield a dosage of, e.g., from about 0.01 to about 50 mg/kg daily, or from about 0.05 to about 20 mg/kg daily, or from about 0.1 to about 10 mg/kg daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compositions may be formulated for rectal administration as a suppository. For parenteral use, including intravenous, intramuscular, intratumoral, intraperitoneal, intranasal, or subcutaneous routes, the nanoparticles, alone or in combination with other active ingredient(s), may be provided in sterile aqueous solutions or suspensions, buffered to an appropriate pH and isotonicity or in parenterally acceptable oil. Suitable aqueous vehicles can include Ringer’s solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1,000 µg/kg/minute of agent admixed with a pharmaceutical carrier over a period ranging from several minutes to several days.

For nasal, inhaled, or oral administration, the nanoparticles, alone or in combination with other active ingredient(s), may be administered using, for example, a spray formulation also containing a suitable carrier.

For topical applications, the nanoparticles, alone or in combination with other active ingredient(s), are preferably formulated as creams or ointments or a similar vehicle suitable for topical administration. For topical administration, the nanoparticles, alone or in combination with other active ingredient(s), may be mixed with a pharmaceutical carrier at a concentration of about 0.1% to about 10% of drug to vehicle. Another mode of administering the nanoparticles, alone or in combination with other active ingredient(s), may utilize a patch formulation to effect transdermal delivery.

In certain embodiments, the present disclosure provides pharmaceutical composition comprising the nanoparticles, alone or in combination with other active ingredient(s), and methylcellulose. In certain embodiments, methylcellulose is in a suspension of about 0.1, 0.2, 0.3, 0.4, or 0.5 to about 1%. In certain embodiments, methylcellulose is in a suspension of about 0.1 to about 0.5, 0.6, 0.7, 0.8, 0.9, or 1%. In certain embodiments, methylcellulose is in a suspension of about 0.1 to about 1%. In certain embodiments, methylcellulose is in a suspension of about 0.1, 0.2, 0.3, 0.4, 0.5, 0.6, 0.7, 0.8, 0.8, or 1%. In certain embodiments, methylcellulose is in a suspension of about 0.5%.

In certain embodiments, “preventative” treatment is meant to indicate a postponement of development of a disease, a symptom of a disease, or medical condition, suppressing symptoms that may appear, or reducing the risk of developing or recurrence of a disease or symptom. In certain embodiments, “curative” treatment includes reducing the severity of or suppressing the worsening of an existing disease, symptom, or condition.

One of ordinary skill in the art may modify the formulations within the teachings of the specification to provide numerous formulations for a particular route of administration. In particular, the nanoparticles, alone or in combination with other active ingredient(s), may be modified to render them more soluble in water or other vehicle. It is also well within the ordinary skill of the art to modify the route of administration and dosage regimen of a particular nanoparticle, alone or in combination with other active ingredient(s), in order to manage the pharmacokinetics of the present compounds for maximum beneficial effect in a patient.

### E. Exemplary Embodiments

In some embodiments, the present disclosure relates to the prevention and/or treatment of diseases or disorders associated with solid tumors. The present disclosure provides for methods, combinations, and pharmaceutical compositions for treating solid tumor(s) in a subject, using, inter alia, an effective amount of nanoparticle comprising a) an inner core comprising a non-cellular biocompatible material, b) and outer surface comprising a cellular membrane derived from a platelet, and c) an immunomodulating agent that is a toll-like receptor (TLR) agonist and/or an upregulator of the opioid growth factor receptor, e.g., resiquimod, for treating such solid tumor(s). Certain exemplary embodiments are described in Bahmani, B., Gong, H., Luk, B.T, et al. Intratumoral immunotherapy using platelet-cloaked nanoparticles enhances antitumor immunity in solid tumors. Nat Commun 12, 1999 (2021). https://doi.org/10.1038/s41467-021-22311-z, the disclosure of which is incorporated herein by reference in its entirety for all purposes.

Nanoparticulate systems that can deliver drugs to a patient in a targeted manner, or that control release of drugs, have been long recognized as beneficial. Nanoparticles in particular have been shown to be capable of localizing to a particular tissue or cell type, thereby reducing the amount of drug in other areas of the body not requiring treatment. This is important when treating a disease such as cancer, where it is imperative to localize treatment to diseased sites in order to reduce toxic, sometimes life-threatening, adverse effects. This is also particularly important in cancer immunotherapy in order to avoid over-activation of the immune system, which would potentially lead to autoimmune diseases or cytokine storms.

Therapeutics that offer such targeted therapy and/or controlled release must also deliver an effective amount of drug. It can be a challenge to prepare nanoparticle systems that balance the size of each nanoparticle (to have advantageous delivery properties) with the amount of drug associated with each nanoparticle. In addition, it is advantageous to increase the nanoparticle residence time at the diseased site in order to maximize the therapeutic effect of the treatment.

Accordingly, a need exists for novel nanoparticle formulations and methods of making such, that can deliver therapeutic levels of drugs to treat cancer, while also simultaneously inducing immunity to such cancers and reducing patient side effects.

In some embodiments, the present disclosure provides for a therapeutic nanoparticle system that includes an active agent for immunomodulation that is a TLR⅞ agonist and a biocompatible polymer. For example, disclosed herein is a therapeutic nanoparticle comprising approximately 1 to 10 weight percent of a therapeutic immunomodulatory agent (such as resiquimod), approximately 50 to 99 weight percent of a biocompatible polymer, and approximately 20 to 50 weight percent of platelet membrane. For example, the biocompatible polymer may be a homopolymer such as poly(lactic) acid homopolymer, or a diblock copolymer such as poly(lactic-co-glycolic) acid.

In some embodiments, the present disclosure provides for a method for treating solid tumors via a tumor-targeting nanoparticle, which method comprises administering, to a subject in need, an effective amount of a nanoparticle comprising a) an inner core comprising a non-cellular material, b) an outer surface comprising a cellular membrane derived from a platelet; and c) an immunomodulating agent that is a TLR⅞ agonist, e.g., resiquimod, for treating such solid tumor.

The diameter of disclosed nanoparticles may be, for example, about 85 to about 140 nm. Disclosed therapeutic nanoparticles may be stable for at least 5 days at -80° C. in a sucrose solution. Disclosed nanoparticles may substantially immediately release about 80% of the therapeutic agent when placed in a phosphate buffer solution at 37° C.

In some embodiments, the present disclosure relates to polymeric nanoparticles that include a therapeutic agent, and methods of making such. In some embodiments, a “nanoparticle” refers to any particle possessing a diameter of less than 1 µm. Disclosed therapeutic nanoparticles may include nanoparticles possessing a diameter of approximately 85 to 140 nm containing approximately 1 to 10 weight percent of an immunomodulating agent that is a TLR agonist (for example: resiquimod, imiquimod, or motolimod). Nanoparticles disclosed herein can include one or more biodegradable polymers.

### Polymers

In some embodiments, disclosed nanoparticles include a matrix of polymers. Disclosed nanoparticles typically include one polymer, e.g., a monopolymer or a diblock co-polymer. Disclosed therapeutic nanoparticles include a therapeutic agent that may be associated with the surface of, encapsulated within, surrounded by, and/or dispersed throughout a polymeric matrix.

A wide variety of polymers and methods for forming nanoparticles are known in the art of nanomedicine and drug delivery. Any polymer can be used in accordance with the present invention. Polymers can be natural or synthetic polymers. Polymers can be homopolymers or copolymers comprising multiple monomers. Proposed polymers may be biocompatible and/or biodegradable. Biocompatibility typically refers to the lack of, or reduced acute rejection of, material by the immune system resulting in toxicity. One simple test to assess biocompatibility can be to expose a polymer to cells in vitro. Non-biocompatible polymers may induce significant cell death at moderate concentrations, while biocompatible polymers will not. Biodegradable typically refers to the ability for a polymer to degrade chemically and/or biologically, within a physiological environment such as the body. Biodegradable polymers are those that, when introduced into cells, are broken down by biologically (e.g., via cellular machinery) or chemically (e.g., via hydrolysis) into components that are biocompatible (e.g., do not pose significant toxic effect on cells).

The term “polymer,” in some embodiments, is given its IUPAC definition, e.g., a molecule of high relative molecular mass, the structure of which essentially comprises the multiple repetition of units derived, actually or conceptually, from molecules of low relative molecular mass. Repeated subunits may be identical, or in some cases, there may be more than one type of repeated subunit present within the polymer. Disclosed particles can include copolymers, which, in some embodiments, describes multiple polymers that have been associated with each, typically by covalent bonding of the respective polymers together.

In some embodiments, polymers may be polyesters, including homopolymers comprising lactic acid units, such as poly-L-lactic acid, poly-D-lactic acid, poly-D,L-lactic acid, poly-L-lactide, poly-D-lactide, and poly-D,L-lactide, collectively referred to herein as “PLA,” as well as copolymers comprising lactic acid and glycolic acid units, such as poly(lactic-co-glycolic) acid and poly(lactide-co-glycolide), collectively referred to herein as “PLGA.” PLA and PLGA are biocompatible and biodegradable polymers.

### Nanoparticles

In some embodiments, disclosed nanoparticles may have a substantially spherical configuration, though the nanoparticles, upon swelling or shrinkage, may adopt a non-spherical configuration. Disclosed nanoparticles can have a diameter of less than 1 µm. In particular embodiments, disclosed nanoparticles have a diameter of approximately 85-140 nm.

Disclosed nanoparticles may have a substantially spherical configuration, though the nanoparticles, upon swelling or shrinkage, may adopt a non-spherical configuration. Disclosed nanoparticles have a diameter of less than 1 µm. In particular embodiments, disclosed nanoparticles have a diameter of approximately 85-140 nm.

In some cases, the interior of the nanoparticle is more hydrophobic than the surface of the particle. For instance, the interior of the particle may be relatively hydrophobic with respect to the surface of the particle, and a drug or other payload may be hydrophobic, and readily loads into the relatively hydrophobic nanoparticle core. The payload can thus be contained within the interior of the particle, which can shield it from the external environment surrounding the nanoparticle. Thus, a payload loaded into a nanoparticle administered into a patient will be protected from a patient’s body, and the body may also be protected from exposure to the payload for at least a period of time.

In one embodiment, the invention comprises a nanoparticle comprising a) an inner core comprising a non-cellular biocompatible material, b) and outer surface comprising a cellular membrane derived from a platelet, and c) an immunomodulating agent that is a TLR⅞ agonist, e.g., resiquimod, for treating such solid tumor.

### Preparation of Nanoparticles

In some embodiments, another aspect of the invention is directed to systems and methods of making the disclosed nanoparticles. In a particular embodiment, the methods described herein form nanoparticles that have a high amount of encapsulated immunotherapeutic agent (1-10 weight percent).

In an embodiment, a nanoemulsion process is provided. For example, a therapeutic agent and a polymer (for example, PLA or PLGA) are mixed with an organic solution to form a first organic phase. Such first phase may include about 5 to about 10% weight solids. The first organic phase may be combined with a first aqueous solution to form a second phase. The organic solution can include, for example, acetonitrile, tetrahydrofuran, ethyl acetate, isopropyl alcohol, isopropyl acetate, dimethylformamide, methylene chloride, dichloromethane, chloroform, acetone, benzyl alcohol, sodium cholate, Tween 80, or the like, and combinations thereof. In an embodiment, the organic phase may include benzyl alcohol, ethyl acetate, dichloromethane, and combinations thereof. The second phase can be between about 1 and 10 weight %. The aqueous solution can be water, optionally in combination with one or more of sodium cholate, ethyl acetate, and benzyl alcohol.

The oil phase may use solvent that is minimally miscible with the aqueous phase. Therefore, when mixed at a low enough ratio and/or when using water pre-saturated with the organic solvents, the oil phase remains liquid. The oil phase may be emulsified into an aqueous solution and, as liquid droplets, sheared into nanoparticles using, for example, high energy dispersion systems, such as homogenizers or sonicators. The aqueous portion of the emulsion, otherwise known as the “water phase”, may be a surfactant solution consisting of sodium cholate and pre-saturated with ethyl acetate and benzyl alcohol or combinations thereof.

Emulsifying the second phase to form an emulsion phase may be performed in one or two emulsification steps. For example, a primary emulsion may be prepared, and then emulsified to form a fine emulsion. The primary emulsion can be formed using simple mixing, high pressure homogenization, probe sonication, stirring, or homogenization via rotor stator. The primary emulsion may be formed into a fine emulsion through the use of probe sonicator or a high pressure homogenizer, e.g., by using 1 or more passes through a homogenizer. For example, when a high pressure homogenizer is used, the pressure used may be about 5,000 to about 15,000 psi.

Solvent evaporation of dilution is needed to complete the extraction of solvent and solidify the particles. An aqueous quench may be used for better control over the kinetics of extraction and a more scalable process. For example, the emulsion can be diluted into cold water to a concentration sufficient to dissolve all organic solvent to form a quenched phase. Quenching may be performed at a temperature of 1-5° C.

The solubilized phase can be filtered to recover the nanoparticles and to remove un-encapsulated drug. Ultrafiltration membranes may be used to concentrate the nanoparticle suspension and remove organic solvent, free drug, and surfactants. Filtration may be performed via a tangential flow filtration (TFF) system. For example, by using a membrane with a pore size suitable to retain nanoparticles while allowing smaller agents to pass, nanoparticles can be selectively separated and concentrated. Exemplary membranes with molecular weight cut-offs of 300-500 kDa may be used. After purifying and concentrating the nanoparticle suspension, the particles may be passed through one or more sterilizing filters.

In exemplary embodiment of preparing nanoparticles, an organic phase is formed, composed of a mixture of a therapeutic agent, e.g., resiquimod, and polymer (PLA). The organic phase may be mixed with an aqueous phase at approximately a 1:4.65 ratio (oil phase:aqueous phase) where the aqueous phase is composed of a surfactant and optionally dissolved solvent. A primary emulsion may then be formed by the combination of the two phases through the use of a rotor stator homogenizer. The primary emulsion is then formed into a fine emulsion through the use of a high pressure homogenizer. Such fine emulsion may then be quenched by addition to deionized water under mixing. An exemplary quench:emulsion ratio may be about approximately 1:1. Formed nanoparticles may then be isolated through either centrifugation or ultrafiltration/diafiltration.

### Membrane Coating

In some embodiments, cellular membrane derived from platelets may be used to coat the disclosed nanoparticles. Platelet membrane has specific biological properties that may allow increased residence time at diseased sites via binding to specific targets at the diseased site. The disclosed nanoparticle therefore may possess platelet-mimicking properties for immunocompatibility and binding/adhesion to biological components.

### F. EXAMPLES

### Example 1. Nanoparticle Preparation - Emulsion Process

In this example, an organic phase is formed, composed of a mixture of a therapeutic agent, e.g., resiquimod, and polymer (PLA). The organic phase is mixed with an aqueous phase at approximately a 1:4.65 ratio (oil phase:aqueous phase) where the aqueous phase is composed of a surfactant (sodium cholate) and some dissolved solvent (7 volume % ethyl acetate). 7% solids in the organic phase is used.

The primary coarse emulsion is formed by the combination of the two phases through the use of a rotor stator homogenizer for 90 seconds at 12,000 rpm. The rotor stator yielded a homogeneous milky solution. The rotor stator is used as the standard method of coarse emulsion formation, although a high speed mixer may be suitable at a larger scale.

The primary emulsion is then formed into a fine emulsion through the use of a high pressure homogenizer. The primary emulsion is passed through the homogenizer twice at 10,000 psi. The size of the coarse emulsion does not significantly affect the particle size after successive passes through the high pressure homogenizer (Microfluidics International Corporation LM-20). The effect of scale on particle size showed scale dependence. The trend shows that larger batches produce smaller particle sizes. Table 1 summarizes exemplary emulsification parameters

TABLE 1

Emulsification Process Parameters

| Parameter | Value |
|---|---|
| Coarse emulsion formation | Rotor stator |
| Homogenizer feed pressure | 10000 psi |
| Interaction chamber | 75 µm Y-chamber |
| Number of homogenizer passes | 2-3 passes |
| Water phase [sodium cholate] | 0.2% |
| W:O ratio | 4.65:1 |
| [Solids] in oil phase | 7% |

The fine emulsion is then quenched by addition to deionized water at 1-5° C. under mixing In the quench unit operation, the emulsion is added to a cold aqueous quench under agitation. This serves to extract a significant portion of the oil phase solvents, effectively hardening the nanoparticles for downstream filtration. Chilling the quench significantly improved drug encapsulation. The quench:emulsion ratio is approximately 1:1. Table 2 summarizes exemplary quench process parameters.

TABLE 2

Quench Process Parameters

| | |
|---|---|
| Parameter | Value |
| Initial quench temperature | 1-5ºC |
| Quench:emulsion ratio | 1:1 |
| Quench hold/processing temp | 1-5ºC |

The nanoparticles are then isolated via a tangential flow filtration process to concentrate the nanoparticle suspension and buffer exchange the solvents, free drug, and surfactants from the solution into water. A regenerated cellulose membrane is used with a molecular weight cutoff (MWCO) of 300 kDa. Table 3 summarizes exemplary TFF parameters used.

TABLE 3

TFF Process Parameters

| | |
|---|---|
| Parameter | Value |
| Membrane Material | Regenerated cellulose |
| MWCO | 300 kDa |
| Flow rate | ^{~}220 mL/min |
| Nanoparticle Concentration | 6 mg/mL |
| Diafiltration Number of Diavolumes | 6 diavolumes |
| Membrane Area | 235 cm² |

After the TFF process, the nanoparticle suspension is passed through a sterilizing filter (0.2 µm). Table 4 summarizes exemplary parameters for exemplary use, as well as exemplary characteristics obtained.

TABLE 4

Summary of Parameters

| Parameter | Value |
|---|---|
| Polymer | PLA |
| Polymer solution | 60 mg/ml in73:27 ethyl acetate:benzyl alcohol |
| Volume of polymer solution | 50 ml |
| Resiquimod amount | 500 mg |
| Emulsification outer phase | 232.5 ml (10 mM tris pH 7.5, 0.2% w/w sodium cholate, 7 vol% ethyl acetate) |
| Coarse emulsion | Homogenized at 12k rpm for 90 s at 1-59C |
| Microfluidizer pressure and passes | 10k psi for 2 passes |
| Quench phase/temperature | 250 ml (10 mM tris pH 7.5) @ 1-5ºC |
| Emulsion:quench ratio | 1:1 |
| Loading | 4.9 wt% |
| Size (Z average) | 86.17 nm |
| Polydispersity index | 0.054 |

### Example 2. In Vitro Release

An in vitro release method is used to determine the initial burst phase release from nanoparticles at 37° C. A dialysis system was designed to maintain sink conditions and to prevent nanoparticles form entering the release samples. The dialysis system is as follows: 3 mL slurry of resiquimod nanoparticles (approximately 5 mg/mL of PLA nanoparticles, corresponding to approximately 250 µg/mL resiquimod concentration) in 8% sucrose is placed in a 20 kDa MWCO dialysis cassette. The cassette is placed into 1 L phosphate buffer with an attached buoy and stir bar, with continual stirring at 150 rpm. The samples are tested for resiquimod concentration prior to dissolution to determine the total dose of resiquimod in each dialysis cassette. At each pre-determined time point, a 1 mL sample is withdrawn from each dissolution vessel and placed into an HPLC vial for analysis by HPLC.

### Example 3. Particle Size Analysis

Particle size is analyzed by dynamic light scattering (DLS). DLS is performed using a Malvern Instruments Nano ZS zetasizer instrument at 25° C. The output from DLS is associated with the hydrodynamic radius of the nanoparticles, which includes the platelet membrane coating.

### Example 4. Intratumoral Immunotherapy Using Platelet-Cloaked Nanoparticles Enhances Antitumor Immunity in Colorectal Adenocarcinoma

Intratumoral immunotherapy is an emerging modality for the treatment of solid tumors that can induce local and systemic antitumor immunity. Toll-like receptor (TLR) agonists have shown promise for eliciting both innate and adaptive immune responses. However, systemic administration of these agonists often results in the development of adverse side effects, thereby limiting their clinical use. Herein, it was investigated whether localized delivery of the TLR agonist, resiquimod (R848), via platelet membrane-coated nanoparticles (PNP-R848) can elicit potent antitumor responses in a colorectal tumor model. The natural membrane coating provides a facile means of enhancing interactions with the tumor microenvironment, thereby maximizing the biological activity of R848 at low drug dosages. As a monotherapy, intratumoral administration of PNP-R848 strongly enhances local immune activation and leads to complete tumor regression in 100% of mice, while providing absolute protection against repeated and aggressive tumor re-challenges. The enhanced ability of the PNP-R848 to mobilize immune cell populations that are critical for antitumor immunity enables it to significantly outperform more traditional R848 formulations. The findings disclosed herein highlight the promise of locally delivering immunostimulatory payloads using biomimetic nanocarriers, which possess advantages such as enhanced biocompatibility and natural targeting affinities that can be leveraged to develop safe and effective treatments against a wide range of solid tumors.

Herein the development of a platelet membrane-cloaked nanoparticle (PNP) for the intratumoral delivery of R848 is reported. The plasma membrane derived from human platelets, with its multitude of proteins, glycoproteins, and lipids, bestows platelet-mimicking properties such as selective adherence to cells in the tumor microenvironment³⁷. Cell membrane coating is a facile approach for improving biocompatibility while enabling nanoparticle platforms to effectively interface with biological targets, such as tumors, through multimodal interactions³⁸. It is demonstrated that R848-loaded PNP (PNP-R848) exhibited prolonged retention at the tumor site and improved cellular interactions within the tumor microenvironment. This enabled the nanoformulation to exert significant biological activity upon intratumoral administration, even at low R848 dosages that would otherwise be ineffective when administered systemically. In an MC38 murine colorectal adenocarcinoma model, it was shown that PNP-R848 promoted the strong activation of APCs within the draining lymph node (DLN) and increased immune infiltration. This ultimately led to a potent antitumor response that facilitated the complete short-term rejection of established tumors while bestowing long-term immunity that protected against repeated and highly aggressive tumor re-challenges.

### Nanoparticle Synthesis and Characterization

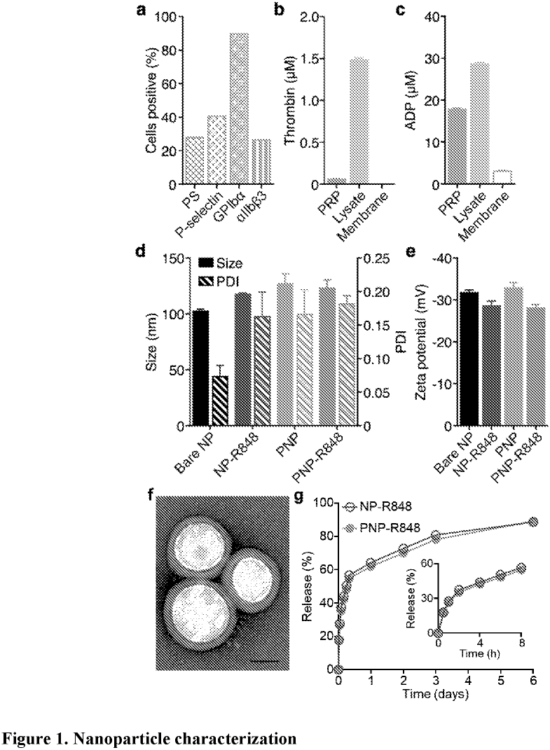

Given the multitude of interactions of platelets with other cell types and tissues³⁹⁻⁴⁴, we aimed to leverage these unique abilities to design a nanoparticle platform incorporating natural targeting abilities. This was done by directly coating the membrane isolated from human platelets through a differential centrifugation and freeze-thaw process onto synthetic polylactic acid (PLA) nanoparticle cores via sonication³⁷. The presence of phosphatidylserine, P-selectin, GPIbα, and integrin αIIbβ3 were confirmed on the surface of the platelet membrane ghosts by flow cytometry (FIG. 1a). Phosphatidylserine, P-selectin, and the full αIIbβ3 complex are expressed on the membrane surface upon platelet activation⁴⁵⁻⁴⁸. GPIbα is responsible for von Willebrand factor-mediated platelet adhesion⁴⁹. P-selectin, αIIbβ3, and GPIbα have all been implicated in cancer pathogenesis, suggesting important interactions with tumor cells⁵⁰. Despite the activation state of the membrane, additional assays for thrombin and adenosine diphosphate verified the successful removal of these platelet-activating molecules responsible for propagating thrombotic responses, thus mitigating safety concerns (FIGS. 1b,c). Physicochemical characterizations revealed that membrane coating slightly increased the size of both the bare PLA nanoparticle cores as well as the bare R848-loaded nanoparticle cores (NP-R848) (FIG. 1d). Additionally, the surface zeta potential was similar between all samples (FIG. 1e). Transmission electron microscopy revealed that the final PNP-R848 formulation possessed a core-shell structure with a layer of membrane coating on the outside (FIG. 1f). Finally, the release of the R848 payload was studied over time, and the profiles for both the bare NP-R848 and coated PNP-R848 formulations matched closely, where more than 80% of the encapsulated payload was released within the first 24 h (FIG. 1g).

### Nanoparticle Interaction With Tumors

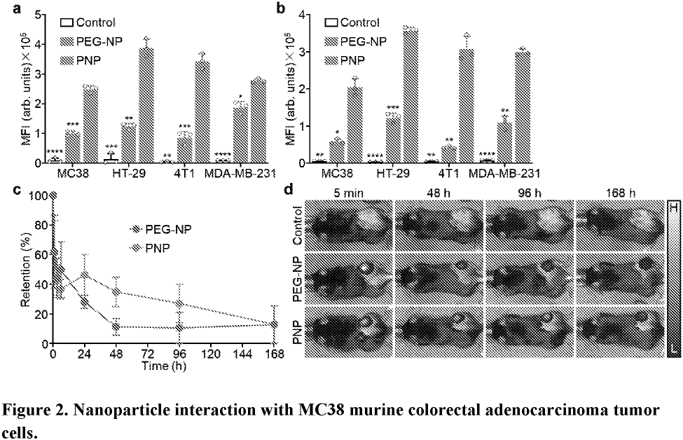

In order to assess the interaction of PNP with solid tumor cell types, both binding and uptake were studied in vitro. Fluorescent dye-labeled nanoparticles were incubated with a panel of murine and human cancer cells, including MC38, HT-29, 4T1, and MDA-MB-231, at 4° C. for the binding study and 37° C. for the uptake study. It was observed by flow cytometry that PNP much more readily bound to all four cancer cells compared with a polyethylene glycol (PEG)-coated nanoparticle (PEG-NP) control (FIG. 2a). These results correlated well with cellular uptake, which was also significantly higher for PNP than for PEG-NP in all of the cell lines (FIG. 2b). Considering the enhanced interaction of PNP with MC38 cells in vitro, the retention time of PNP in an MC38 tumor model in vivo was tested next. After allowing the tumors to establish, mice received a single intratumoral administration of dye-labeled PEG-NP or PNP, and the nanoparticles were tracked using a live imaging system over the course of 7 days (FIGS. 2c,d). Initially, there was a similar drop in the amount of nanoparticles present within the tumor. As time progressed, the difference between the two groups increased, and the greatest contrast was observed at 48 h, where on average 35% of the PNP remained, while only 11% of the PEG-NP was retained within the tumor. Taken together, these studies demonstrate that the platelet membrane coating, which displays surface markers known to play a role in cancer cell binding⁵¹, was able to significantly increase nanoparticle affinity to the MC38 tumor cells compared with a more traditional PEG coating.

### In Vitro Immunostimulatory Activity

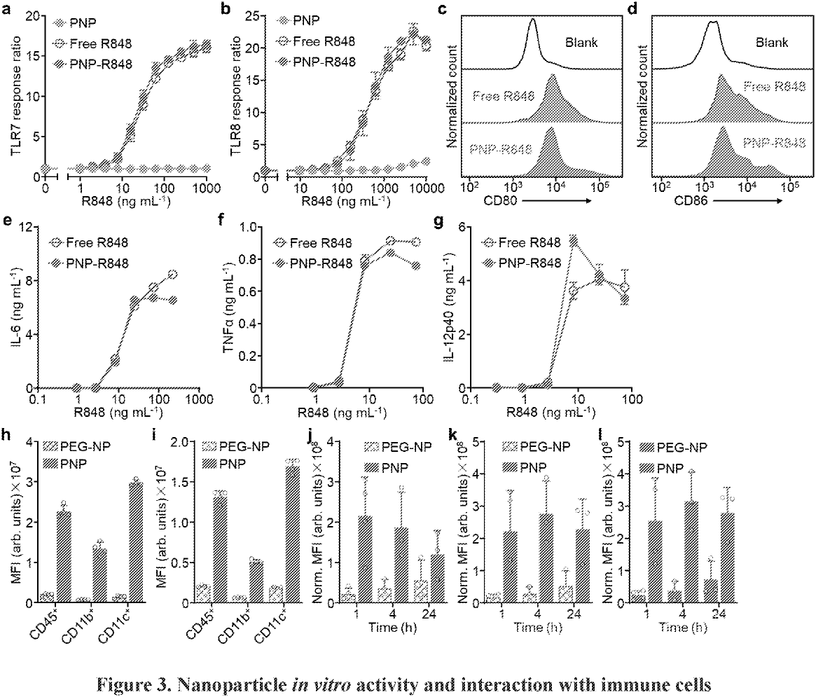

To directly assess the biological activity of the R848 payload, PNP-R848 was incubated with human reporter cell lines expressing either TLR7 or TLR8, which provide a colorimetric readout in response to NF-_{K}B activation (FIGS. 3a,b). The cells were incubated with free R848 or PNP-R848 for 21 h, and results showed that the activities of the two were roughly equivalent at the same drug concentration. As expected, PNP nanoparticles without drug loading showed minimal TLR7 and TLR8 activation. Next, the biological effect of PNP-R848 on bone marrow-derived cells (BMDCs) was studied, and it was observed that the formulation could induce the upregulation of CD80 and CD86, two APC maturation markers that serve as co-stimulatory signals for mediating downstream immune responses (FIGS. 3c,d). The expression levels of CD80 and CD86 were comparable to those induced by free R848, indicating that the loading of the payload into the nanoparticles did not affect their potent immunomodulatory activity. Additionally, the ability of PNP-R848 to elicit the production of proinflammatory cytokines such as IL-6, tumor necrosis factor α (TNFα), and IL-12 by BMDCs was assessed (FIGS. 3e-g). After incubation with various concentrations of free R848 or PNP-R848, the culture supernatant was analyzed by enzyme-linked immunosorbent assays (ELISAs). For each cytokine that was studied, results showed a dose-dependent release pattern that was similar for both samples. Empty PNP, regardless of whether the platelet membrane was sourced from humans or mice, did not induce appreciable APC maturation or cytokine secretion; this supports the notion that the immune response elicited by PNP-R848 was driven largely by inclusion of the R848 payload^{52,53}.

### Nanoparticle Interaction With Immune Cells

Next, the interactions of the PNP formulation with various BMDC subpopulations were evaluated (FIGS. 3h,i). PNP showed a significant increase in both cell binding and uptake as compared to PEG-NP for all cell subtypes examined, including CD45⁺ leukocytes, CD11b⁺ macrophages, and CD11c⁺ dendritic cells. It is believed that the enhanced uptake of PNP by BMDCs may have contributed to the increased cytokine release observed in the preceding study. The in vivo interaction of the nanoformulation with tumor cell populations at various timepoints after intratumoral administration of dye-labeled PEG-NP and PNP (FIG. 3j-1) was next studied. Overall, the uptake of PNP by the total population of cells in the tumor was significantly higher, evidenced by a significant increase in fluorescence intensity compared to PEG-NP. When immune cell subsets in the tumor were evaluated, higher uptake for PNP was also observed among CD45⁺ leukocytes and CD11c⁺ dendritic cells across all timepoints.

### Antitumor Efficacy in a Mouse Tumor Model

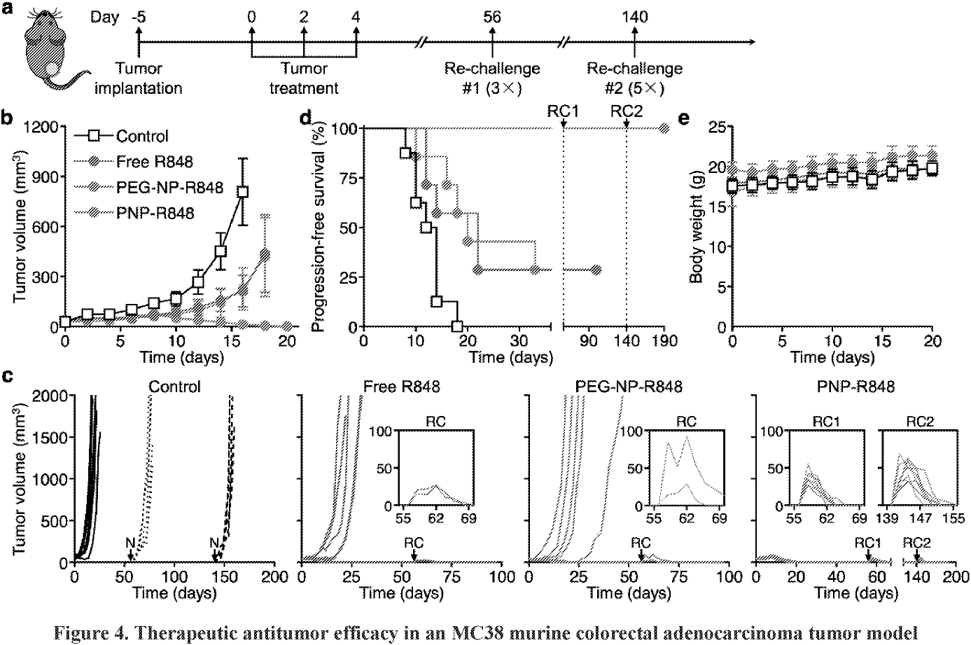

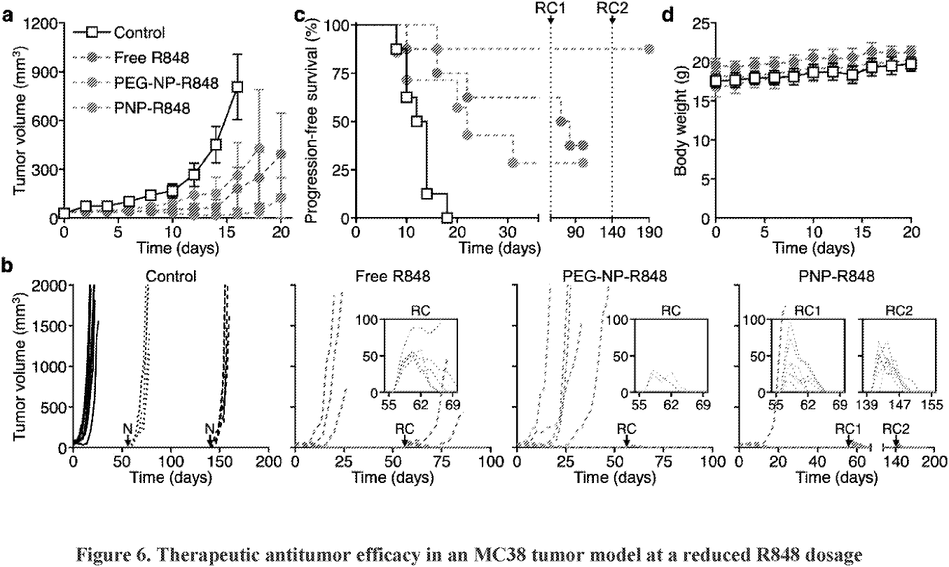

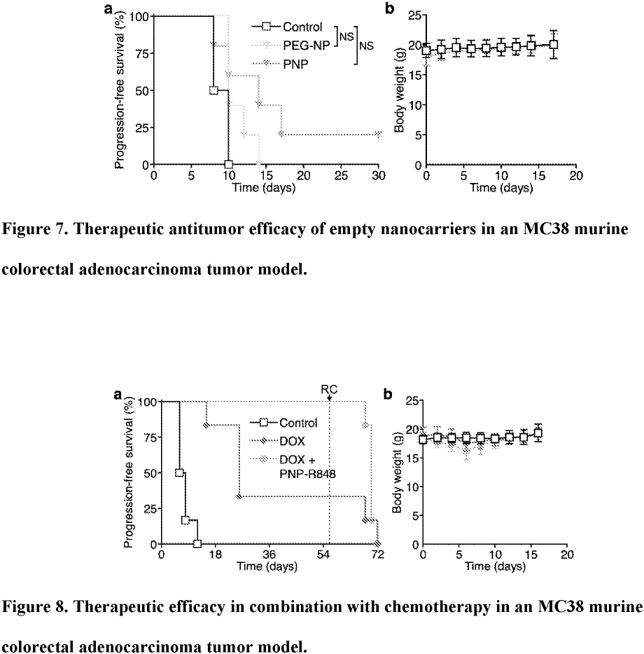

The antitumor efficacy of PNP-R848 was evaluated using an MC38 murine colon adenocarcinoma model in immunocompetent C57BL/6 mice (FIG. 4a). Each animal received a subcutaneous injection of 1 × 10⁶ MC38 cells in the right flank, and the average tumor size was allowed to reach ~30-40 mm³. At this point, the mice started receiving one of the following treatments: 8% sucrose as a negative control, free R848, PEG-NP loaded with R848 (PEG-NP-R848), or PNP-R848, each at a drug dosage of 15 µg per injection. Treatments were administered intratumorally every other day for a total of 3 times, after which the mice were monitored regularly to assess therapeutic efficacy (FIGS. 4b-e). Rapid regression followed PNP-R848 treatment, and complete tumor eradication was observed for 100% of the mice. Tumor growth was considerably delayed when treating with either free R848 or PEG-NP-R848, but there was significant disease progression in a majority of the mice by approximately 30 days after the start of treatment. In the end, both free R848 and PEG-NP-R848 treatments yielded a 28.6% long-term survival rate. Treatment efficacy was also evaluated when reducing the drug dosage by 2.5-fold to 6 µg of R848 per injection (FIG. 6). In this case 87.5% of mice treated with PNP-R848 completely rejected the tumor challenge, and 28.6% of mice survived after treatment with PEG-NP-R848. Interestingly, free R848 at the lower dosage outperformed the corresponding higher dosage treatment, with a 62.5% long-term survival rate. None of the treatments had a significant impact on the weight of the mice, suggesting that there was no acute toxicity. It should also be noted that neither PEG-NP nor PNP without any R848 loading had a statistically significant impact on the progression-free survival of the mice (FIG. 7).

In order to determine if the surviving animals had developed long-term immunity against MC38 cancer cells, the mice were re-challenged with a 3-fold higher inoculum subcutaneously into the right flank 56 days after initiation of the first treatment (FIGS. 4c,d). For the survivors that had been treated with either dose of PNP-R848, the second tumor challenge was rejected at a 100% rate. Though animals treated with the 6-µg dosage of free R848 initially demonstrated a 62.5% survival rate, the overall survival dropped to 37.5% after the tumor re-challenge, indicating inefficient development of an adaptive immune response against MC38 cells. The remainder of the surviving animals in the other groups all rejected the re-challenge, with no tumor progression observed at least 100 days after the start of the initial treatment. These results show that, while free R848 exhibits antitumor activity, it is not as efficient as the PNP-R848 formulation at eliciting long-lasting immunity. Notably, mice that were treated with PNP-R848 all rejected a second re-challenge with a 5-fold higher dose of cancer cells performed 140 days after initial treatment (FIGS. 4c,d).

Therapeutic efficacy of PNP-R848 in combination with chemotherapy was also assessed (FIG. 8). While free doxorubicin administered intratumorally at a high dosage of 63 µg prolonged survival, the improvement was modest compared with PNP-R848 treatment. When combining the two treatment modalities, 100% of the mice survived the initial tumor challenge, although a greater than 10% loss in body weight 6 days after the start of treatment suggested the presence of toxicity. Despite some promising initial results, all mice treated with doxorubicin, both with or without PNP-R848, succumbed after re-challenge with a 3-fold higher dose of cancer cells. Given that leukodepletion is often a side effect of chemotherapy⁵⁴, these results highlight the need for an intact immune response to achieve long-lasting antitumor protection.

### Effect of Treatment on Immune Cell Populations in Vivo

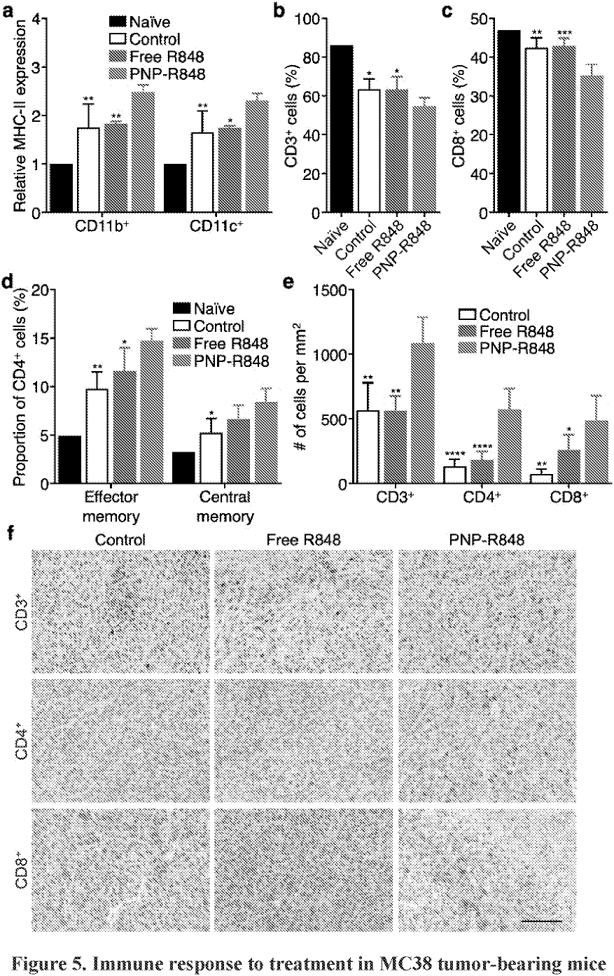

To elucidate the immune responses associated with treatment efficacy, the DLN from tumor-bearing mice were collected 7 days after administration of low dose free R848 or PNP-R848 on the same schedule as above. PNP-R848 was able to significantly elevate the expression of major histocompatibility complex II (MHC-II), a maturation marker, on CD11b⁺ and CD11c⁺ APC subsets (FIG. 5a). No significant differences in MHC-II expression were observed in the same cell populations after free R848 treatment. Interestingly, the overall percentage of CD3⁺ T cells in the DLN on day 7 dropped in response to PNP-R848 treatment (FIG. 5b), and this also held true for the proportion of CD8⁺ T cells (FIG. 5c). Among the T cells that were present, the CD4⁺ population had a significantly elevated proportion with the effector memory (CD44^{hi}CD62L^{low}) and central memory (CD44^{hi}CD62L^{hi}) phenotypes (FIG. 5d). Since a drop in the percentage of T cells in the DLN was observed, whether this was due to their migration into the tumor was then assessed. The tumor tissue was histologically sectioned and stained for various immune cell subsets (FIGS. 5e,f). Indeed, increased densities of both CD4⁺ and CD8⁺ T cells were found in the tumors of mice treated with PNP-R848 compared with free R848. Overall, the data indicate that PNP-R848, by improving tissue retention, was able to enhance the stimulation of APCs in the DLN, resulting in better priming of T cells and their subsequent recruitment into the tumor. This ultimately led to tumor eradication and the generation of memory T cells to fight subsequent tumor re-challenge.

### Antitumor Efficacy in a Mouse Breast Cancer Model

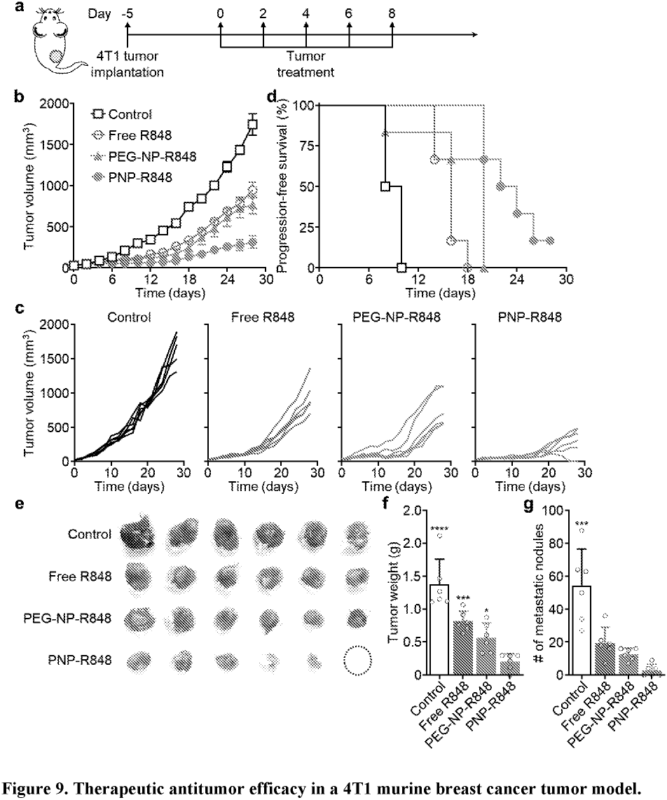

To further evaluate the applicability of PNP-R848 as a generalizable treatment against solid tumors, anticancer efficacy was tested in a syngeneic murine 4T1 triple-negative breast cancer model established using BALB/c mice (FIG. 9a). Each animal was subcutaneously implanted with 5 × 10⁵ tumor cells in the right flank, and the average tumor size was allowed to reach ~30-40 mm³ before treatment with either 8% sucrose, free R848, PEG-NP-R848, or PNP-R848 at a drug dosage of 15 µg per injection. The mice were treated every other day for a total of 5 times, and the tumor sizes and progression-free survival were monitored (FIGS. 9b-d). Similar to the MC38 model, administration of PNPR848 resulted in significant inhibition of 4T1 tumor growth. With PNP-R848 treatment, progression-free survival was prolonged to 23 days, compared to 9 days for the control group. Both free R848 and PEG-NP-R848 exhibited an intermediate level of antitumor efficacy. This trend was also reflected on day 30 after the first treatment, when the tumors were excised and weighed (FIGS. 9e,f). Notably, PNP-R848 had a marked effect on the number of metastatic nodules in the lungs, reducing the average number per lung to 3 nodules from more than 50 for the control group (FIG. 9g).

### Discussion

Here, a novel biomimetic delivery vehicle to locally retain a potent immunomodulator at tumor sites is reported. The TLR7 family of agonists stimulates dendritic cell activation and subsequent T cell priming, which leads to tumor-specific T cell immune responses and immunity^{33,55,56}. There are some reports demonstrating that systemic administration of R848 via the intravenous or intraperitoneal route promotes antitumor immune responses, but high drug dosages are generally required to achieve therapeutic efficacy^{18,57}. In one case, mice bearing MC38 tumors were administered with a total of 600 µg R848, yet complete tumor regression was not observed⁵⁷. In contrast, it has been shown that local delivery of R848 at more modest dosages, sometimes in combination with chemotherapy, can lead to complete tumor regression and long-term protective immunity^{58,59}. It should be noted that considerable efficacy has generally been observed for intratumoral R848 only when combining with other immunostimulatory agents^{59,60}. The data suggest that the biomimetic platelet-derived membrane increased the interaction of PNP-R848 with various cells in the tumor microenvironment, thereby enhancing the bioavailability of R848 at the tumor site and surrounding lymphoid tissue after local delivery. This enabled significant reduction of the required dose of R848 compared to previous studies while maintaining its therapeutic potential. Even at a low total dosage of 18 µg per mouse, complete remission was observed in almost all mice receiving PNP-R848 alone in an MC38 colorectal tumor model.

Not wishing to be bound by any particular theory, it is hypothesized that PNP-R848 administered intratumorally is able to effect tumor regression by triggering a local inflammatory response and activating the resident APCs, some of which can migrate to the DLN and promote a subsequent influx of primed T cells into the tumor tissue. Infiltration of cytotoxic CD8⁺ cells into the tumor was observed as early as 7 days post-treatment, which corresponded to the beginning of tumor size reduction in the efficacy study. This data corroborate recent findings that intratumoral activation of TLR⅞ transforms the tumor microenvironment and induces immune cell infiltration into the tumor³². Additionally, the observed increase in effector and central memory T cells in the DLN supports the development of systemic adaptive antitumor immunity. When re-challenged with more aggressive tumor implantation protocols, the animals that had eradicated the initial MC38 tumors following intratumoral treatments with PNP-R848 exhibited strong immunity and completely rejected the new implants within 2 weeks. Notably, significant reduction in lung metastasis was also observed in a 4T1 breast cancer model after PNP-R848 treatment.

In conclusion, a biomimetic nanoformulation has been developed that leverages platelet membrane coating to enhance the delivery and retention of an immunostimulatory payload for intratumoral cancer immunotherapy. The membrane-coated nanoparticles efficiently interacted with cancer cells, leading to enhanced tumor retention in vivo and maximizing the activity of the encapsulated R848 payload. In an immunocompetent murine model of colorectal cancer, treatment with PNP-R848 was able to completely eradicate tumor growth, leading to long-term antitumor immunity that allowed all of the surviving mice to reject a subsequent re-challenge. The potent activity of the formulation was further corroborated in a murine model of triple-negative breast cancer, where significant reduction in metastasis was achieved. This approach for the localized delivery of small molecule immunomodulators could be easily applied across a wide range of solid tumor types, providing a meaningful strategy for eliciting potent immune responses that could greatly enhance patient outcomes in the clinic.

### Methods

Platelet membrane preparation and characterization. Human platelet-rich plasma (PRP) was obtained from the San Diego Blood Bank. To collect the platelet membrane, the PRP was first diluted 2× with buffer consisting of 140 mM NaCl (Fisher Chemical), 2.7 mM KCl (Fisher Chemical), 3.8 mM 4-(2-hydroxyethyl)-1-piperazineethanesulfonic acid (HEPES; Acros), 5 mM ethylene glycol-bis(β-aminoethyl ether)-N,N,N′,N′-tetraacetic acid (Bioworld), and 2 µM prostaglandin E1 (PGE1; AdooQ BioScience), followed by centrifugation at 2,000 g for 15 min with no brake. The supernatant was removed and the platelets were resuspended with a lysis buffer containing a mixture of 75 mM NaCl, 6 mM NaHCO₃ (Fisher Chemical), 1.5 mM KCl, 0.17 mM Na₂HPO₄ (Fisher Chemical), 0.5 mM MgCl₂ (Alfa Aesar), 20 mM HEPES, 1 mM ethylenediaminetetraacetic acid (Fisher Chemical), 1 µM PGE1, 0.01% NP40 surfactant (Boston Bioproducts), and protease inhibitors (Thermo Scientific). The platelet membrane was derived by a repeated freeze-thaw process. The platelet mixture was frozen at -80° C., thawed at room temperature, and pelleted by centrifugation at 21,100 g for 10 min. The pellet was then resuspended in the lysis buffer, and the freeze-thaw was repeated two more times. After the repeated washes, the membrane was suspended in water for coating onto the nanoparticle cores.

Quantification of total membrane protein concentration was performed using a Pierce BCA protein assay kit (Life Technologies). Flow cytometry was used to probe for the expression of specific surface markers on the platelet membrane using FITC-conjugated annexin V (Biolegend), Alexa488-conjugated anti-human P-selectin (AK4; Biolegend), Alexa647-conjugated anti-human GPIbα (HIP1; Biolegend), and Alexa647-conjugated anti-human αIIbβ3 (PAC-1; Biolegend). The probes were incubated with purified platelet membrane in phosphate-buffered saline (PBS; Gibco) for 30 min in the dark at room temperature. After incubation, the membrane was washed by centrifugation at 21,100 g. Data was collected using a Becton Dickinson Accuri C6 flow cytometer and analyzed with Flowjo software.

Nanoparticle synthesis and physicochemical characterization. R848-loaded nanoparticles were synthesized using a single emulsion process. First, polylactic acid (PLA; R202H; Evonik) and R848 (BOC Sciences) were dissolved in an organic phase consisting of benzyl alcohol (Acros) and ethyl acetate (Fisher Chemical) at concentrations of 60 mg/mL and 10 mg/mL, respectively. The mixture was then added to 5× volume of ice-cold outer phase media consisting of 10 mM Tris pH 7.5 (Invitrogen) with 0.2 wt% sodium cholate (Alfa Aesar) and 7 vol% ethyl acetate. This solution was homogenized at 12,000 rpm for 90 s using a Kinematica Polytron PT 3100 homogenizer before being passed through a Microfluidics LM20 Microfluidizer (outfitted with a Y chamber) three times. This mixture was then added to an equal volume of outer phase media, and the solvent was evaporated overnight in a fume hood while stirring at 200 rpm. Unloaded nanoparticle cores were fabricated using the same procedure without R848 in the organic phase. Platelet membrane coating was performed by sonication of the R848-loaded or unloaded nanoparticle cores with platelet membrane at a polymer to membrane mass ratio of 1:0.7. Polyethylene glycol (PEG)-coated nanoparticles were fabricated using the same procedure as for the nanoparticle cores, but using PEG-conjugated PLA (PolySciTech) to replace 10 wt% of the unconjugated PLA. To prepare nanoparticles loaded with 1,1′-dioctadecyl-3,3,3′,3′-tetramethylindodicarbocyanine (DiD; Biotium), the dye was added to a 10 mg/mL PLA solution in acetone at 0.1 wt% of the polymer. Then, 2 mL of this solution was added dropwise to 4 mL of water to form the dye-loaded nanoparticle cores. After overnight solvent evaporation, the nanoparticles were coated with platelet membrane by sonication. Hydrodynamic nanoparticle size and surface zeta potential were measured by dynamic light scattering using a Malvern Zetasizer Nano ZS. For imaging, the nanoparticles were stained with 0.2 wt% uranyl acetate (Electron Microscopy Sciences) and visualized with an FEI Tecnai Spirit G2 BioTWIN transmission electron microscope.

Drug loading and release. R848 loading was analyzed using a reverse-phase ultra-high-performance liquid chromatography (UHPLC) method. The UHPLC system consisted of a binary gradient pump, in-line degasser, autosampler and Thermo Scientific Vanquish photodiode array detector. Separation and quantitative analysis of R848 were achieved on a 3.5 µm Waters XBridge™ C18 column (2.1 × 150 mm) with the mobile phase flowing at a rate of 1.0 mL/min and a detection wavelength of 227 nm. Mobile phase A consisted of 10 mM sodium phosphate (Fisher Chemical) with 0.1% triethylamine (Acros) and pH adjusted to 2.45, while mobile phase B consisted of 100% acetonitrile (Fisher Chemical). The acquisition run time for each analysis was 6.5 min with a gradient consisting of 15% mobile phase B from 0 to 3 min, 45% mobile phase B from 3 to 5 min and 15% mobile phase B from 5.1 to 6.5 min. The samples were first diluted in acetonitrile and then diluted in a combination of 30% acetonitrile and 70% 0.1 N hydrochloric acid (Acros). They were then injected into the column after a series of six standard injections prepared by diluting R848 in 100% acetonitrile. Drug release kinetics of PNP-R848 were performed in PBS with 0.05% Triton X-100 (Alfa Aesar) utilizing 20 kDa dialysis cassettes (Thermo Scientific). Reconstituted samples were transferred to the dialysis cassettes via a syringe with a 21-gauge needle. The dissolution experiments were run at 37° C. while stirring at 200 rpm for 72 h. Samples were withdrawn at various timepoints and analyzed neat by UHPLC.

In vitro binding and uptake of PNP by murine and human cancer cells. MC38 murine colon adenocarcinoma cells (Kerafast) and MDA-MB-231 human mammary gland adenocarcinoma cells (HTB-26; American Type Culture Collection) were cultured in Dulbecco’s modified Eagle’s medium (Gibco) supplemented with 10% fetal bovine serum (Corning). 4T1 murine mammary gland cancer cells (CRL2539; American Type Culture Collection) were cultured in RPMI-1640 medium (Gibco) supplemented with 10% fetal bovine serum. HT-29 human colorectal adenocarcinoma cells (HTB-38; American Type Culture Collection) were cultured in McCoy’s 5a medium (Gibco) supplemented with 10% fetal bovine serum. For the binding study, DiD-loaded PNP or PEG-NP were incubated with 5 × 10⁵ MC38, HT-29, 4T1, or MDA-MB-231 cells in 100 µL of media. The final nanoparticle concentration for this incubation was 0.2 mg/mL. The incubation was performed for 30 min at 4° C. in order to minimize endocytic uptake, after which the cells were washed 3 times with PBS and examined using flow cytometry. For the uptake study, the incubation was instead performed for 10 min at 37° C. Data were collected using a Becton Dickinson Accuri C6 flow cytometer and analyzed with FlowJo software.

TLR activation assays. HEK-Blue hTLR7 and HEK-Blue hTLR8 reporter cells (Invivogen) were cultured as directed by the manufacturer. For the dose-response experiments, 20 µL of PNP, free R848, or PNP-R848 were loaded into 96-well cell culture plates at 10× the desired final concentration (0.977 to 1000 ng/mL). The cultured reporter cells were rinsed with warm PBS, resuspended in 1 mL warm PBS, and then detached from culture flasks by gentle scraping. The cells were diluted to a concentration of 2.2 × 10⁵ cells/mL in HEK-Blue detection medium (Invivogen), whereupon 180 µL of the cell suspension was immediately added to the sample dilutions. Absorbance at 655 nm was measured after 21 h of incubation at 37° C. in 5% CO₂.

Nanoparticle activity on BMDCs. All animal experiments were performed in accordance with guidelines approved by the Institutional Animal Care and Use Committee of the University of California San Diego. Female C57BL/6 mice were euthanized via CO₂ asphyxiation. Intact tibias were isolated from each mouse, dip briefly into 70% ethanol, and stored in RPMI cell culture media (Gibco) over ice. Both ends of each tibia were cut and each bone was flushed with 10 mL of RPMI using a syringe attached with a 23-gauge needle. Bone marrow cells were collected and washed by centrifugation at 320 g for 9 min. Finally, cells were passed through a 50-µm cell strainer (Corning). For cytokine release and co-stimulatory marker characterization, BMDC cells were counted, and 500,000 cells were plated per well in 6-well plates. Various concentrations of free R848 and PNP-R848 were added to the cells and incubated at 37° C. for 24 h. Afterwards, the supernatant was assayed for IL-6 release using an OptEIA mouse IL-6 ELISA kit (BD Biosciences). The cells were washed and scraped from the plates, followed by staining with FITC-conjugated anti-mouse CD45 (30-F11; BD Biosciences), PE-conjugated anti-mouse CD80 (16-10A1; BD Biosciences), and APC-conjugated anti-mouse CD86 (GL-1; Biolegend). Data was collected using a Becton Dickinson Accuri C6 flow cytometer and analyzed with Flowjo software.

BMDC binding and uptake. For cell binding studies, DiD-loaded PNP or PEG-NP were incubated with 1 × 10⁶ BMDC cells in 100 µL media at a final nanoparticle concentration of 0.2 mg/mL. Incubation was performed for 30 min at 4° C., after which the cells were washed three times with PBS and subsequently stained using FITC-conjugated anti-mouse CD45, PE-conjugated anti-mouse CD11b (M1/70; Biolegend), and PE/Cy7-conjugated anti-mouse CD11c (N418; Biolegend). For uptake studies, incubation was performed for 10 min at 37° C. Data was collected using a Becton Dickinson Accuri C6 flow cytometer and analyzed with Flowjo software.

In vivo interaction with tumors. To develop tumors, 1 × 10⁶ MC38 cells were implanted subcutaneously into the right flank of 6-week old female C57BL/6 mice. Tumor volumes were calculated using the equation: volume = (length x width²)/2. For the tumor retention study, the average tumor size was allowed to reach 100 mm³, after which the mice were administered with 8% sucrose as the negative control (n = 3), DiD-labeled PNP (n = 3), and DiD-labeled PEG-NP (n = 3). Each animal received one intratumoral injection and was imaged using a Xenogen IVIS 200 system at various timepoints, including 5 min and 1, 3, 6, 24, 48, 96, and 168 h with the same acquisition time and filter settings. Acquired images were analyzed by the IVIS software to quantify the fluorescence intensity of the tumors and to determine tumor retention percentage.

To assess interaction with immune cells, mice with tumors with an average volume of 100 mm³ were intratumorally administered with DiD-labeled PNP or PEG-NP. At 1, 4, and 24 h, groups of mice were euthanized, and the tumor tissue was processed into single cell suspensions by digesting in a solution containing collagenase IV (Sigma-Aldrich) and DNase type IV (Sigma-Aldrich) at final concentrations of 1 mg/mL and 10 µg/mL, respectively. The cells were stained using FITC-conjugated anti-mouse CD45 and PE/Cy7-conjugated anti-mouse CD11c. Data was collected using a Becton Dickinson Accuri C6 flow cytometer and analyzed with Flowjo software.

Therapeutic efficacy in a murine MC38 tumor model. Mice were implanted with 1 × 10⁶ MC38 cells subcutaneously into the right flank, which were allowed to grow to an average size of ~30-40 mm³. The mice were then intratumorally treated every other day for a total of 3 times. The treatment groups included: 8% sucrose (n = 7), free R848 (n = 7), PEG-NP-R848 (n = 7), and PNP-R848 (n = 8). Each treatment group received 15 µg of R848 per treatment. The injection volume was 30 µL for all the treatments, and delivery was done via syringe with a 31-gauge needle. Tumor growth and mouse weight were monitored every other day. Progression-free survival was defined as tumor volume < 200 mm³. All the mice that rejected the initial MC38 inoculum were re-challenged subcutaneously using 3 × 10⁶ MC38 cells on day 56 after the start of the first treatment. Mice in the PNP-R848 treatment group that were tumor-free at the end of the initial re-challenge study were re-challenged a second time with 5 × 10⁶ MC38 cells on day 140. For each re-challenge, 5 naive C57BL/6 mice that received the same MC38 tumor cell challenge were used as controls to verify tumorigenicity.

In vivo immune profiling. Mice bearing MC38 tumors were treated on the same schedule as the antitumor efficacy study with 8% sucrose (n = 3), low dose free R848 (n = 4), and low dose PNP-R848 (n = 4). The mice were then euthanized 7 days after the first treatment, and the inguinal draining lymph node (DLN) (on the same side as the tumor) was processed into a single cell suspension by shearing the tissue using a 50 \-µm cell strainer. Cells were stained with different antibodies, including BV510-conjugated anti-mouse CD3 (17A2; Biolegend), FITC-conjugated anti-mouse CD4 (RM4-5; eBiosciences), APC/Cy7-conjugated anti-mouse CD8 (53-6.7; Invitrogen), PerCP/Cy5.5-conjugated anti-mouse CD62L (MEL-14; eBioscience), APC-conjugated anti-mouse CD44 (IM7; BD Biosciences), V500-conjugated anti-mouse CD45 (30-F11; BD Biosciences), APC-conjugated anti-mouse MHC-II (M5/114.15.2; Tonbo Bioscience), APC/Cy7-conjugated anti-mouse CD11b (M1/70; BD Biosciences), and PE/Cy7-conjugated anti-mouse CD11c. Data was collected using a Becton Dickinson Accuri C6 flow cytometer and analyzed with Flowjo software. Tumor tissues were fixed in formalin (Fisher Scientific) for 24 h and were then transferred into 70% ethanol prior to histological sectioning by the Moores Cancer Center Tissue Technology Shared Resource. Tumor sections were stained for mouse CD3, CD4, and CD8 using AEC substrate and counterstained with Mayer’s Hematoxylin. Slides were imaged with a Hamamatsu Nanozoomer 2.0HT slide scanner.

Therapeutic efficacy in a murine MC38 tumor model at a reduced R848 dosage. Mice were implanted with 1 × 10⁶ MC38 cells subcutaneously into the right flank, which were allowed to grow to an average size of ~30-40 mm³. The mice were then intratumorally treated every other day for a total of 3 times. The treatment groups included: 8% sucrose (n = 7), free R848 (n = 8), PEG-NP-R848 (n = 7), and PNP-R848 (n = 8). Each treatment group received 6 µg of R848 per treatment. The injection volume was 30 µL for all the treatments, and delivery was done via syringe with a 31-gauge needle. Tumor growth and mouse weight were monitored every other day. Progression-free survival was defined as tumor volume < 200 mm³. All the mice that rejected the initial MC38 inoculum were re-challenged subcutaneously using 3 × 10⁶ MC38 cells on day 56 after the start of the first treatment. Mice in the PNP-R848 treatment group that were tumor-free at the end of the initial re-challenge study were re-challenged a second time with 5 × 10⁶ MC38 cells on day 140. For each re-challenge, 5 naive C57BL/6 mice that received the same MC38 tumor cell challenge were used as controls to verify tumorigenicity.

Therapeutic efficacy with unloaded nanocarriers. Mice were implanted with 1 × 10⁶ MC38 cells subcutaneously into the right flank, which were allowed to grow to an average size of ~30-40 mm³. The mice received intratumoral treatments every other day for a total of four times. The treatment groups included: 8% sucrose (n = 5), PEG-NP (n = 5), and PNP (n = 5). The nanoparticles used in this study were empty and did not contain R848. Tumor growth and mouse weight were monitored every other day. Progression-free survival was defined as tumor volume < 200 mm³.

Therapeutic efficacy in combination with doxorubicin. Mice were implanted with 1 × 10⁶ MC38 cells subcutaneously into the right flank, which were allowed to grow to an average size of ~30-40 mm³. The mice received intratumoral treatments every other day for a total of 3 times. The treatment groups included: 8% sucrose (n = 6), free doxorubicin (n = 6), and doxorubicin + PNP-R848 (n = 6). Mice received 63 µg of doxorubicin and 15 µg of R848 per dose. For the combination treatment, doxorubicin and PNP-R848 were mixed pre-administration and the animal received one intratumoral injection containing both. Tumor growth and mouse weight were monitored every other day. Progression-free survival was defined as tumor volume < 200 mm³. All the mice that rejected the initial MC38 inoculum were re-challenged subcutaneously using 3 × 10⁶ MC38 cells on day 56 after the start of the first treatment. Five naive C57BL/6 mice that received the same MC38 tumor cell challenge were used as controls to verify tumorigenicity.

Therapeutic efficacy in a murine 4T1 tumor model. Female BALB/c mice (Charles River Laboratories) were implanted with 5 × 10⁵ 4T1 cells subcutaneously into the right flank, which were allowed to grow to an average size of ~30-40 mm³. The mice were then treated every other day for a total of 5 treatments. The treatment groups included: 8% sucrose (n = 6), free R848 (n = 6), PEG-NP-R848 (n = 6), and PNP-R848 (n = 6). Each group received 15 µg of R848 per treatment. The injection volume was 30 µL for all the treatments, and delivery was done via syringe with a 31-gauge needle. Tumor growth and mouse weight were monitored every other day. Progression-free survival was defined as tumor volume < 200 mm³. The study was terminated 30 days after the first treatment, and the tumors and lungs were harvested. For metastatic nodule counting, lung tissues were fixed using Bouin’s solution (Election Microscopy Sciences).

### REFERENCES

References are listed below:
1 Tumeh, P. C. et al. PD-1 blockade induces responses by inhibiting adaptive immune resistance. Nature 515, 568-571, doi:10.1038/nature13954 (2014).
2 Vilain, R. E. et al. Dynamic changes in PD-L1 expression and immune infiltrates early during treatment predict response to PD-1 blockade in melanoma. Clin. Cancer Res. 23, 5024-5033, doi:10.1158/1078-0432.CCR-16-0698 (2017).
3 Sharma, P., Wagner, K., Wolchok, J. D. & Allison, J. P. Novel cancer immunotherapy agents with survival benefit: Recent successes and next steps. Nat. Rev. Cancer 11, 805-812, doi:10.1038/nrc3153 (2011).
4 Mihara, K. et al. Activated T-cell-mediated immunotherapy with a chimeric receptor against CD38 in B-cell non-Hodgkin lymphoma. J. Immunother. 32, 737-743, doi:10.1097/CJI.0b013e3181adaffl (2009).
5 Buchbinder, E. & Hodi, F. S. Cytotoxic T lymphocyte antigen-4 and immune checkpoint blockade. J. Clin. Invest. 125, 3377-3383, doi:10.1172/JCI80012 (2015).
6 Chen, L. & Han, X. Anti-PD-⅟PD-L1 therapy of human cancer: Past, present, and future. J. Clin. Invest. 125, 3384-3391, doi:10.1172/jci80011 (2015).
7 Grupp, S. A. et al. Chimeric antigen receptor-modified T cells for acute lymphoid leukemia. N. Engl. J. Med. 368, 1509-1518, doi:10.1056/NEJMoa1215134 (2013).
8 Newick, K., O’Brien, S., Moon, E. & Albelda, S. M. CAR T cell therapy for solid tumors. Annu. Rev. Med. 68, 139-152, doi:10.1146/annurev-med-062315-120245 (2017).
9 Michot, J. M. et al. Immune-related adverse events with immune checkpoint blockade: A comprehensive review. Eur. J. Cancer 54, 139-148, doi:10.1016/j.ejca.2015.11.016 (2016).
10 Kluger, H. M. et al. PD-L1 studies across tumor types, its differential expression and predictive value in patients treated with immune checkpoint inhibitors. Clin. Cancer Res. 23, 4270-4279, doi:10.1158/1078-0432.CCR-16-3146 (2017).
11 Liu, C., Han, C. & Liu, J. The role of Toll-like receptors in oncotherapy. Oncol. Res. 27, 965-978, doi:10.3727/096504019X15498329881440 (2019).
12 Matijevic, T. & Pavelic, J. Toll-like receptors: Cost or benefit for cancer? Curr. Pharm. Des. 16, 1081-1090, doi:10.2174/138161210790963779 (2010).
13 Medzhitov, R. Toll-like receptors and innate immunity. Nat. Rev. Immunol. 1, 135-145, doi:10.1038/35100529 (2001).
14 Braunstein, M. J., Kucharczyk, J. & Adams, S. Targeting Toll-like receptors for cancer therapy. Target. Oncol. 13, 583-598, doi:10.1007/s11523-018-0589-7 (2018).
15 Lester, S. N. & Li, K. Toll-like receptors in antiviral innate immunity. J. Mol. Biol. 426, 1246-1264, doi:10.1016/j.jmb.2013.11.024 (2014).
16 Vasilakos, J. P. & Tomai, M. A. The use of Toll-like receptor ⅞ agonists as vaccine adjuvants. Expert Rev. Vaccines 12, 809-819, doi:10.1586/14760584.2013.811208 (2013).
17 Aranda, F. et al. Trial watch: Toll-like receptor agonists in oncological indications. Oncoimmunology 3, e29179, doi:10.4161/onci.29179 (2014).
18 Michaelis, K. A. et al. The TLR⅞ agonist R848 remodels tumor and host responses to promote survival in pancreatic cancer. Nat. Commun. 10, 4682-4682, doi:10.1038/s41467-019-12657-w (2019).
19 Schön, M. P. & Schön, M. TLR7 and TLR8 as targets in cancer therapy. Oncogene 27, 190-199, doi:10.1038/sj.onc.1210913 (2008).
20 Dovedi, S. J. et al. Systemic delivery of a TLR7 agonist in combination with radiation primes durable antitumor immune responses in mouse models of lymphoma. Blood 121, 251-259, doi:10.1182/blood-2012-05-432393 (2013).
21 Cheadle, E. J. et al. A TLR7 agonist enhances the antitumor efficacy of obinutuzumab in murine lymphoma models via NK cells and CD4 T cells. Leukemia 31, 1611-1621, doi:10.1038/leu.2016.352 (2017).
22 Nishii, N. et al. Systemic administration of a TLR7 agonist attenuates regulatory T cells by dendritic cell modification and overcomes resistance to PD-L1 blockade therapy. Oncotarget 9, 13301-13312, doi:10.18632/oncotarget.24327 (2018).
23 Rodell, C. B. et al. TLR⅞-agonist-loaded nanoparticles promote the polarization of tumour-associated macrophages to enhance cancer immunotherapy. Nat. Biomed. Eng. 2, 578-588, doi:10.1038/s41551-018-0236-8 (2018).
24 Dummer, R. et al. An exploratory study of systemic administration of the Toll-like receptor-7 agonist 852A in patients with refractory metastatic melanoma. Clin. Cancer Res. 14, 856-864, doi:10.1158/1078-0432.CCR-07-1938 (2008).
25 Harrison, L., Astry, C., Kumar, S. & Yunis, C. Pharmacokinetics of 852A, an imidazoquinoline Toll-like receptor 7-specific agonist, following intravenous, subcutaneous, and oral administrations in humans. J. Clin. Pharmacol. 47, 962-969, doi:10.1177/0091270007303766 (2007).
26 Pockros, P. J. et al. Oral resiquimod in chronic HCV infection: Safety and efficacy in 2 placebo-controlled, double-blind phase IIa studies. J. Hepatol. 47, 174-182, doi:10.1016/j.jhep.2007.02.025 (2007).
27 Savage, P. et al. A phase I clinical trial of imiquimod, an oral interferon inducer, administered daily. Br. J. Cancer 74, 1482-1486, doi:10.1038/bjc.1996.569 (1996).
28 Goldstein, D. et al. Administration of imiquimod, an interferon inducer, in asymptomatic human immunodeficiency virus-infected persons to determine safety and biologic response modification. J. Infect. Dis. 178, 858-861, doi:10.1086/515343 (1998).
29 Gunzer, M. et al. Systemic administration of a TLR7 ligand leads to transient immune incompetence due to peripheral-blood leukocyte depletion. Blood 106, 2424-2432, doi:10.1182/blood-2005-01-0342 (2005).
30 Currie, A. J. et al. Targeting the effector site with IFN-αβ-inducing TLR ligands reactivates tumor-resident CD8 T cell responses to eradicate established solid tumors. J. Immunol. 180, 1535-1544, doi:10.4049/jimmunol.180.3.1535 (2008).
31 Schmidt, C. Immune system’s Toll-like receptors have good opportunity for cancer treatment. J. Natl. Cancer Inst. 98, 574-575, doi:10.1093/jnci/djj198 (2006).
32 Mullins, S. R. et al. Intratumoral immunotherapy with TLR⅞ agonist MEDI9197 modulates the tumor microenvironment leading to enhanced activity when combined with other immunotherapies. J. Immunother. Cancer 7, 244-244, doi:10.1186/s40425-019-0724-8 (2019).
33 Singh, M. et al. Effective innate and adaptive antimelanoma immunity through localized TLR⅞ activation. J. Immunol. 193, 4722-4731, doi:10.4049/jimmunol.1401160 (2014).
35 Trujillo, J. A., Sweis, R. F., Bao, R. & Luke, J. J. T cell-inflamed versus non-T cell-inflamed tumors: A conceptual framework for cancer immunotherapy drug development and combination therapy selection. Cancer Immunol. Res. 6, 990-1000, doi:10.1158/2326-6066.CIR-18-0277 (2018).
36 Dehaini, D., Fang, R. H. & Zhang, L. Biomimetic strategies for targeted nanoparticle delivery. Bioeng. Transl. Med. 1, 30-46, doi:10.1002/btm2.10004 (2016).
37 Hu, C. M. et al. Nanoparticle biointerfacing by platelet membrane cloaking. Nature 526, 118-121, doi:10.1038/nature15373 (2015).
38 Fang, R. H., Kroll, A. V., Gao, W. & Zhang, L. Cell membrane coating nanotechnology. Adv. Mater. 30, 1706759, doi:10.1002/adma.201706759 (2018).
39 Born, G. V. & Cross, M. J. The aggregation of blood platelets. J. Physiol. 168, 178-195, doi:10.1113/jphysiol.1963.sp007185 (1963).
40 Fitzgerald, J. R., Foster, T. J. & Cox, D. The interaction of bacterial pathogens with platelets. Nat. Rev. Microbiol. 4, 445-457, doi:10.1038/nrmicro1425 (2006).
41 Haemmerle, M., Stone, R. L., Menter, D. G., Afshar-Kharghan, V. & Sood, A. K. The platelet lifeline to cancer: Challenges and opportunities. Cancer Cell 33, 965-983, doi:10.1016/j.ccell.2018.03.002 (2018).
42 Kieffer, N. & Phillips, D. R. Platelet membrane glycoproteins: Functions in cellular interactions. Annu. Rev. Cell Biol. 6, 329-357, doi:10.1146/annurev.cb.06.110190.001553 (1990).
43 McEver, R. P. The clinical significance of platelet membrane glycoproteins. Hematol. Oncol. Clin. North Am. 4, 87-105, doi:10.1016/S0889-8588(18)30507-0 (1990).
44 Xu, X. R., Yousef, G. M. & Ni, H. Cancer and platelet crosstalk: Opportunities and challenges for aspirin and other antiplatelet agents. Blood 131, 1777-1789, doi:10.1182/blood-2017-05-743187 (2018).
45 Csongradi, E. et al. Increased levels of platelet activation markers are positively associated with carotid wall thickness and other atherosclerotic risk factors in obese patients. Thromb. Haemost. 106, 683-692, doi:10.1160/TH11-01-0030 (2011).
46 Kannan, M., Ahmad, F. & Saxena, R. Platelet activation markers in evaluation of thrombotic risk factors in various clinical settings. Blood Rev. 37, 100583, doi:10.1016/j.blre.2019.05.007 (2019).
47 Saleh, H. M., Attia, E. A., Onsy, A. M., Saad, A. A. & Abd Ellah, M. M. Platelet activation: A link between psoriasis per se and subclinical atherosclerosis — A case— control study. Br. J. Dermatol. 169, 68-75, doi:10.1111/bjd.12285 (2013).
48 Durrant, T. N., van den Bosch, M. T. & Hers, I. Integrin αIIbβ3 outside-in signaling. Blood 130, 1607-1619, doi:10.1182/blood-2017-03-773614 (2017).
49 Resendiz, J. C., Feng, S., Ji, G. & Kroll, M. H. von Willebrand factor binding to platelet glycoprotein Ib-IX-V stimulates the assembly of an α-actinin-based signaling complex. J. Thromb. Haemost. 2, 161-169, doi:10.1111/j.1538-7836.2003.00497.x (2004).
50 Gay, L. J. & Felding-Habermann, B. Contribution of platelets to tumour metastasis. Nat. Rev. Cancer 11, 123-134, doi:10.1038/nrc3004 (2011).
51 Stone, J. P. & Wagner, D. D. P-selectin mediates adhesion of platelets to neuroblastoma and small cell lung cancer. J. Clin. Invest. 92, 804-813, doi:10.1172/JCI116654 (1993).
52 Kroll, A. V. et al. Nanoparticulate delivery of cancer cell membrane elicits multiantigenic antitumor immunity. Adv. Mater. 29, 1703969, doi:10.1002/adma.201703969 (2017).
53 Zhuang, J. et al. Nanoparticle delivery of immunostimulatory agents for cancer immunotherapy. Theranostics 9, 7826-7848, doi:10.7150/thno.37216 (2019).
54 Bally, M. B., Nayar, R., Masin, D., Cullis, P. R. & Mayer, L. D. Studies on the myelosuppressive activity of doxorubicin entrapped in liposomes. Cancer Chemother. Pharmacol. 27, 13-19, doi:10.1007/BF00689270 (1990).
55 Chi, H. et al. Anti-tumor activity of Toll-like receptor 7 agonists. Front. Pharmacol. 8, 304, doi:10.3389/fphar.2017.00304 (2017).
56 Drobits, B. et al. Imiquimod clears tumors in mice independent of adaptive immunity by converting pDCs into tumor-killing effector cells. J. Clin. Invest. 122, 575-585, doi:10.1172/JCI61034 (2012).
57 Schmid, D. et al. T cell-targeting nanoparticles focus delivery of immunotherapy to improve antitumor immunity. Nat. Commun. 8, 1747, doi:10.1038/s41467-017-01830-8 (2017).
58 Stathopoulos, A. et al. Development of immune memory to glial brain tumors after tumor regression induced by immunotherapeutic Toll-like receptor ⅞ activation. OncoImmunology 1, 298-305, doi:10.4161/onci.19068 (2012).
59 Nie, Y. et al. Development of a curative therapeutic vaccine (TheraVac) for the treatment of large established tumors. Sci. Rep. 7, 14186, doi:10.1038/s41598-017-14655-8 (2017).
60 Da Silva, C. G. et al. Co-delivery of immunomodulators in biodegradable nanoparticles improves therapeutic efficacy of cancer vaccines. Biomaterials 220, 119417, doi:10.1016/j.biomaterials.2019.119417 (2019).

1. A nanoparticle, which comprises:
a) an inner core comprising a non-cellular material;
b) an outer surface comprising a cellular membrane derived from a platelet; and
c) an immunomodulating agent that is a toll-like receptor (TLR) agonist and/or an upregulator of the opioid growth factor receptor.

2. The nanoparticle of claim 1, wherein the inner core comprises a polymer.

3-9. (canceled)

10. The nanoparticle of claim 1, wherein the inner core comprises a biocompatible or a synthetic material selected from the group consisting of poly(lactic-co-glycolic acid) (PLGA), polylactic acid (PLA), polyglycolic acid (PGA), polycaprolactone (PCL), polylysine, and polyglutamic acid.

11. The nanoparticle of claim 1, wherein the cellular membrane comprises a plasma membrane derived from a platelet.

12-16. (canceled)

17. The nanoparticle of claim 1, wherein the immunomodulating agent is a toll-like receptor (TLR) agonist.

18-19. (canceled)

20. The nanoparticle of claim 1, wherein the immunomodulating agent is resiquimod, imiquimod, or motolimod.

21-25. (canceled)

26. The nanoparticle of claim 1, wherein the inner core supports said outer surface.

27-38. (canceled)

39. A process for making a nanoparticle comprising:
a) contacting an immunomodulating agent that is a toll-like receptor (TLR) agonist and/or an upregulator of the opioid growth factor receptor with a polymer to form an organic phase in an organic solvent;
b) contacting said organic phase with an aqueous phase to form a primary emulsion;
c) subjecting said primary emulsion to sonication or a high pressure homogenization to form a fine emulsion;
d) removing said organic solvent from said fine emulsion to form a nanoparticle comprising said immunomodulating agent and said polymer in said fine emulsion; and
e) recovering said nanoparticle from said fine emulsion.

40-69. (canceled)

70. A method for treating or preventing a neoplasm in a subject in need comprising administering to said subject an effective amount of a nanoparticle of claim 1.

71-74. (canceled)

75. The method of claim 70, wherein the subject is a human.

76. The method of claim 70, wherein the immunomodulating agent is a toll-like receptor (TLR) agonist or an upregulator of the opioid growth factor receptor.

77. The method of claim 70, wherein the immunomodulating agent is resiquimod, imiquimod, or motolimod.

78. (canceled)

79. The method of claim 70, wherein the neoplasm is a lymphoma, a leukemia, a brain cancer, glioma/glioblastoma (GBM), a multiple myeloma, a pancreatic cancer, a liver cancer, a stomach cancer, a breast cancer, a kidney cancer, a lung cancer, non-small cell lung cancer (NSCLC), a colorectal cancer, a colon cancer, a prostate cancer, an ovarian cancer, a cervical cancer, a skin cancer, an esophageal cancer, or a head and neck cancer.

80. The method of claim 70, wherein the neoplasm is a solid cancer or tumor.

81-82. (canceled)

83. The method of claim 70, wherein the nanoparticle, medicament delivery or pharmaceutical composition is administered intratumorally or in situ to a solid cancer or tumor site in a subject.

84. (canceled)

85. The method of claim 70, wherein the nanoparticle, medicament delivery or pharmaceutical composition is administered to a subject in combination with another anti-neoplastic agent or substance, e.g., doxycycline.

86. (canceled)

87. The method of claim 70, wherein the nanoparticle, medicament delivery or pharmaceutical composition is administered to a subject that has been treated with anti-neoplastic agent or substance, e.g., doxycycline.

88. (canceled)

89. The method of claim 70, wherein the nanoparticle, medicament delivery or pharmaceutical composition is administered to a subject as a first line of treatment.

90. The method of claim 70, wherein the nanoparticle, medicament delivery or pharmaceutical composition is administered to a subject with relapse neoplasm, e.g., a relapse solid cancer or tumor.

91. (canceled)

92. The method of claim 70, which achieves a survival rate of at least about 80% in the treated subjects for at least about three months.
